(19) United States
(12) Patent Application Publication
(10) Pub. No.: US 2026/0253217 A1
(43) Pub. Date: Aug. 27, 2026

(54) SYSTEM

(71) Applicant: SoftBank Group Corp., Tokyo (JP)

(72) Inventor: Eiichiro KONDO, Tokyo (JP)

(73) Assignee: SoftBank Group Corp., Tokyo (JP)

(21) Appl. No.: 19/539,002

(22) Filed: Feb. 13, 2026

(30) Foreign Application Priority Data
Feb. 21, 2025 (JP) 2025-027024

(51) Int. Cl. G06T 7/00

(52) U.S. Cl. CPC G06T 7/0012

(57) ABSTRACT

The system according to the embodiment comprises an imaging unit, an analysis unit, a determination unit, and a provision unit. The imaging unit captures a facial photograph of a user. The analysis unit analyzes the facial photograph captured by the imaging unit. The determination unit makes a determination using medical data based on the state of the face analyzed by the analysis unit. The provision unit provides the determination result obtained by the determination unit to the user.

### CROSS-REFERENCE TO RELATED APPLICATION

The present application claims priority to and incorporates by reference the entire contents of Japanese Patent Application No. 2025-027024 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult for users to easily grasp their physical condition and health status.

### SUMMARY OF THE INVENTION

The system according to the embodiment comprises an imaging unit, an analysis unit, a determination unit, and a provision unit. The imaging unit captures a facial photograph of a user. The analysis unit analyzes the facial photograph captured by the imaging unit. The determination unit makes a determination using medical data based on the state of the face analyzed by the analysis unit. The provision unit provides the determination result obtained by the determination unit to the user.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

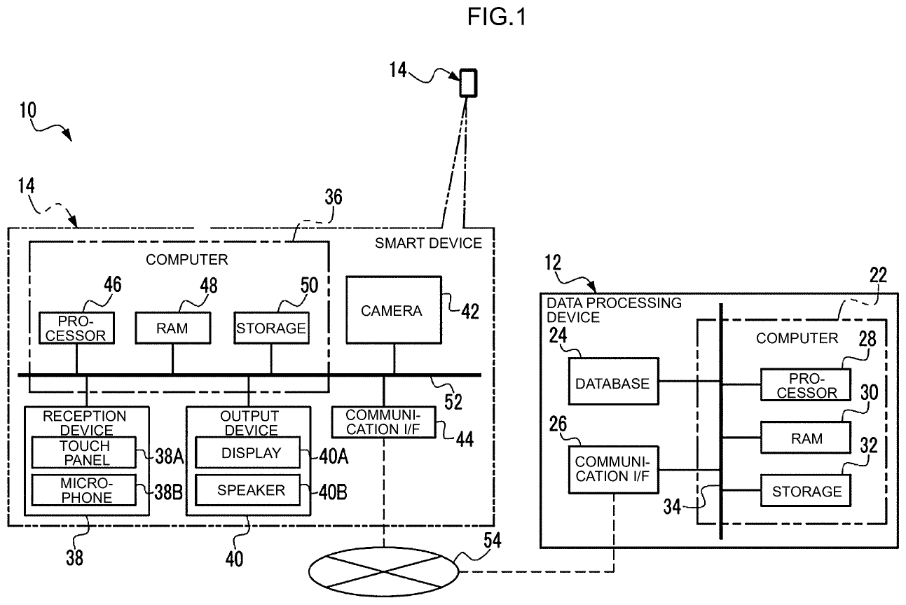

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

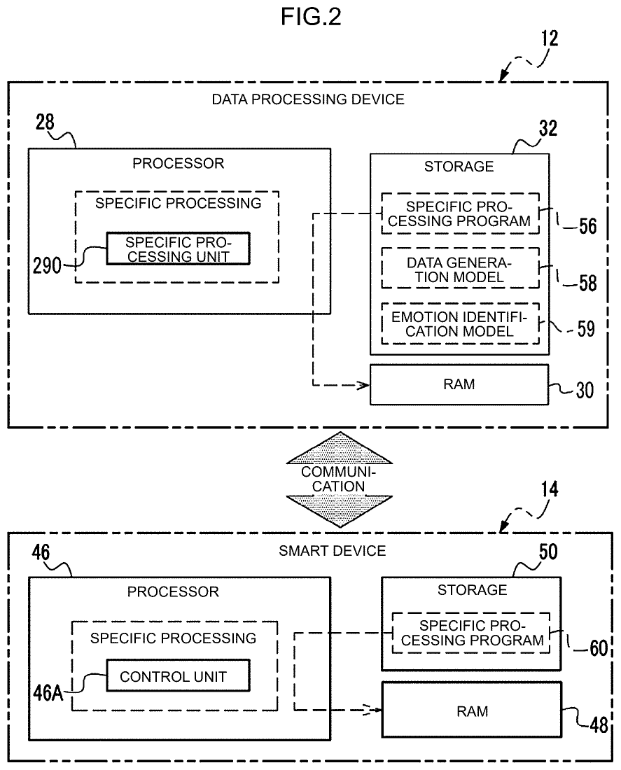

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### EXAMPLE OF THE EMBODIMENT

The health condition determination system according to the embodiment of the present invention is a service that allows users to easily grasp their physical condition and health status for the day, just as they would check the weather, news, or traffic information. In this health condition determination system, the user takes a facial photograph of themselves with a camera, and AI automatically recognizes features such as complexion, swelling, puffiness, wrinkles, and scars. The AI makes determinations based on data accumulated in medical settings (linking the state of the face and diseases), and returns the results to the user. Furthermore, follow-up is performed via services within electronic commerce sites or messenger apps. For example, the system can inform the user of a nearby gym using a MAP function, or provide health recipes through a health recipe provision service. First, the user takes a facial photograph of themselves with a camera. At this time, it is important to ensure that the entire face is clearly captured. For example, shooting under natural light makes it easier to accurately recognize the color and condition of the face. Next, the AI analyzes the captured facial photograph. The AI automatically recognizes features such as complexion, swelling, puffiness, wrinkles, and scars. For example, if the complexion is pale or there are dark circles under the eyes, information related to health status is extracted. The AI determines the correlation between the state of the face and diseases based on data accumulated in medical settings. For example, if the complexion is pale, the system may determine a possibility of anemia, and if swelling is present, it may suggest kidney problems. In this way, the AI determines the user's health status and returns the result to the user. Furthermore, follow-up is performed via services within electronic commerce sites or messenger apps. For example, the MAP function can be used to inform the user of a nearby gym, or the health recipe provision service can be used to provide health recipes. This enables the user to take concrete actions according to their health status. Through this service, users can routinely grasp their health status and take necessary measures. For example, if they feel unwell, they can immediately visit a medical institution. Also, by taking concrete actions to maintain health, they can help prevent diseases and promote health. Thus, the health condition determination system enables users to routinely grasp their health status and take necessary measures. Specifically, this health condition determination system starts processing by having the user take a facial photograph using the camera function of a smartphone or tablet. The system acquires the facial photograph data as an RGB image tensor (e.g., 224×224×3 pixels), and performs preprocessing such as face region detection (e.g., MTCNN or YOLO-based face detection algorithms). The system normalizes the detected face region and applies brightness/contrast correction and noise removal (e.g., Gaussian filter) to improve the input quality for the AI model. The system inputs the facial image tensor into image recognition models such as convolutional neural networks (CNN) or Vision Transformers. For example, the initial layers of the CNN extract low-level features such as edges, hue, brightness, and saturation, while the intermediate layers extract local shape features such as swelling, puffiness, wrinkles, and scars. In the final layer, the system uses fully connected layers or softmax layers to output multiple health-related feature quantities such as complexion (e.g., pale, redness, jaundice), swelling (e.g., cheek expansion degree), puffiness (e.g., left-right asymmetry), wrinkles (e.g., depth, number), and scars (e.g., location, size) as a multidimensional vector. As a specific example, the system generates score vectors such as “complexion: pale (score 0.85)”, “swelling: moderate (score 0.65)”, “wrinkles: deep (score 0.72)”, etc. The system inputs these feature vectors into the determination unit, which uses a machine learning model (e.g., random forest, SVM, or multilayer perceptron) trained on paired data of facial images and diagnostic results accumulated in medical settings (e.g., more than 100,000 facial images and diagnostic labels) to infer the correlation between the state of the face and disease risk. The determination unit outputs probability distributions such as “anemia risk: high (probability 0.78)”, “kidney disease risk: medium (probability 0.52)”, etc. The system performs threshold determination (e.g., high risk for values above 0.7) and rule-based branching on these output values to determine the notification content and subsequent actions for the user. The system passes the determination result to the provision unit, which automatically generates services such as gym search, health recipe recommendation, and medical institution guidance according to the user's current location information and health status via electronic commerce site APIs and messenger app APIs. For example, in health recipe recommendation, the system prioritizes recipes rich in iron for users with high anemia risk, and in gym guidance, proposes low-impact exercise facilities for users with kidney disease risk. Because the system executes this series of processes fully automatically and at high speed, it achieves significant improvements in determination accuracy, processing speed, and user experience compared to conventional manual visual determination and manual search. As a technical effect, the system achieves improved accuracy in health condition estimation through automatic extraction of high-dimensional image features and linkage with medical databases, immediate provision of user-optimized services, and efficiency in data management and communication load. Specific application fields include health management apps for general consumers, remote medical support, corporate health management support, risk assessment for insurance companies, and health monitoring in nursing care settings.

The health condition determination system according to the embodiment comprises an imaging unit, an analysis unit, a determination unit, and a provision unit. The imaging unit captures a facial photograph of the user. For the user's facial photograph, it is important, for example, to ensure that the entire face is clearly captured. For instance, shooting under natural light makes it easier to accurately recognize the color and condition of the face. The analysis unit analyzes the facial photograph captured by the imaging unit. The analysis unit automatically recognizes features such as complexion, swelling, puffiness, wrinkles, and scars. For example, if the complexion is pale or there are dark circles under the eyes, information related to health status is extracted. The determination unit makes a determination using medical data based on the state of the face analyzed by the analysis unit. Medical data may include, for example, hospital medical records, health checkup results, genetic information, and the like. The determination unit determines the correlation between the state of the face and diseases based on data accumulated in medical settings. For example, if the complexion is pale, the system may determine a possibility of anemia, and if swelling is present, it may suggest kidney problems. The provision unit provides the determination result obtained by the determination unit to the user. For example, the provision unit may provide a health recipe by utilizing a health recipe provision service based on the determination result. In addition, the provision unit may inform the user of a nearby gym by utilizing an electronic commerce site based on the determination result. Thus, the health condition determination system according to the embodiment can analyze a facial photograph of the user, determine the health status using medical data, and provide the result. Specifically, the health condition determination system uses a camera module of a smartphone or tablet as the imaging unit, and incorporates automatic exposure control and face detection algorithms (e.g., Haar Cascade, MTCNN) to guide optimal imaging conditions in real time so that the user's entire face is clearly captured. The analysis unit receives the facial image from the imaging unit as an RGB tensor (e.g., 256×256×3), and performs preprocessing such as face region extraction, histogram equalization, and noise removal. The analysis unit uses image recognition models such as convolutional neural networks (CNN) or Vision Transformers to extract feature quantities such as complexion (hue, brightness, saturation), swelling (contour expansion degree), puffiness (left-right asymmetry), wrinkles (depth, number), and scars (location, size) as a multidimensional vector. The analysis unit outputs scores such as “complexion: pale (score 0.82)”, “swelling: mild (score 0.41)”, etc. The determination unit receives the feature vector from the analysis unit and uses a machine learning model (e.g., random forest, SVM, deep learning model) trained on paired data of facial images and diagnostic results accumulated in medical settings (e.g., more than 100,000 cases) to infer the correlation between the state of the face and disease risk. The determination unit outputs probability distributions such as “anemia risk: high (probability 0.76)”, “kidney disease risk: medium (probability 0.54)”, and performs threshold determination and rule-based branching. The provision unit receives the determination result and automatically generates and presents recipes and gym information according to the user's health status via health recipe provision service APIs and electronic commerce site APIs. For example, if the anemia risk is high, the system prioritizes recipes rich in iron, and if the kidney disease risk is present, it prioritizes low-impact exercise facilities. This series of processes is executed fully automatically and at high speed, achieving significant improvements in determination accuracy, processing speed, and user experience compared to conventional manual visual determination and manual search. As a technical effect, the system achieves improved accuracy in health condition estimation through automatic extraction of high-dimensional image features and linkage with medical databases, immediate provision of user-optimized services, and efficiency in data management and communication load. Specific application fields include health management apps for general consumers, remote medical support, corporate health management support, risk assessment for insurance companies, and health monitoring in nursing care settings.

The provision unit can provide a health recipe by utilizing a health recipe provision service based on the determination result. Health recipes may include, for example, nutritional balance, calorie calculation, cooking procedures, and the like, but are not limited to such examples. For instance, the provision unit provides a nutritionally balanced recipe according to the user's health status. The provision unit can also perform calorie calculation and propose an appropriate calorie intake for the user's health status. Furthermore, the provision unit provides detailed cooking procedures and supports the user so that they can easily cook. Thus, by providing health recipes based on the determination result, the provision unit can support the user's health maintenance. Specifically, the provision unit receives the health condition determination result (e.g., labels or probability values such as “anemia risk: high”, “kidney disease risk: medium”) from the determination unit as input, and links with a health recipe database (e.g., structured data including recipe ID, nutrient information, calories, cooking time, allergen information, etc.). The provision unit executes SQL queries or vector searches according to the determination result, and extracts recipe candidates under conditions such as “high iron content”, “low salt”, “high protein, low fat”, etc. The provision unit ranks the extracted recipe candidates using a recommendation engine (e.g., collaborative filtering, content-based recommendation, or Transformer-based recipe recommendation model), taking into account the user's past preference data and allergy information. The provision unit automatically generates and presents detailed information of the finally selected recipe (e.g., ingredient list, quantities, cooking procedures, nutrition table, calorie calculation results) on the user interface, with images or videos. For example, for users with high anemia risk, the system prioritizes recipes such as “iron-fortified spinach and beef recipe (350 kcal, 8 mg iron)”, and for users with kidney disease risk, it prioritizes recipes such as “low-salt, high-potassium elimination recipe (280 kcal, 1.2 g salt)”. Furthermore, the provision unit can display cooking procedures step-by-step and automatically generate voice guides or video tutorials. This enables users to instantly obtain recipes optimized for their health status and take actions directly linked to daily dietary improvement. As a technical effect, the provision unit achieves improved accuracy in personalized recipe recommendation through automatic linkage between health condition determination results and the recipe database, improved user experience, and efficiency in dietary management. Specific application fields include health management apps, remote nutrition guidance, corporate health management support, and dietary management in nursing care settings.

The provision unit can inform the user of a nearby gym by utilizing an electronic commerce site based on the determination result. Information about nearby gyms may include, for example, distance, facilities, fees, business hours, and the like, but is not limited to such examples. For instance, the provision unit proposes the gym closest to the user's current location. The provision unit can also provide information about gym facilities, fees, and business hours, supporting the user in selecting the optimal gym. Thus, by informing the user of a nearby gym based on the determination result, the provision unit can support the user's health maintenance. Specifically, the provision unit receives the health condition determination result (e.g., labels or probability values such as “high risk of lack of exercise”, “medium risk of kidney disease”) and the user's current location information (e.g., GPS coordinates, address data) as input. The provision unit links with electronic commerce sites and gym information databases (e.g., structured data including gym ID, location information, facility list, fee structure, business hours, congestion status, etc.), and extracts gym candidates within a certain distance (e.g., within a 2 km radius) from the user's current location using spatial search algorithms (e.g., KD-Tree, Ball Tree, etc.). The provision unit filters gym facility conditions (e.g., low-impact exercise equipment, rehabilitation facilities, pool, etc.), business hours, and fee plans according to the determination result, and ranks gyms optimal for the user's health status. For example, for users with kidney disease risk, the system prioritizes low-impact exercise facilities, and for users with high risk of lack of exercise, it prioritizes beginner-friendly gyms or facilities offering personal training. The provision unit automatically generates and presents detailed information of the finally selected gym (e.g., name, address, map link, facility photos, fee table, business hours, congestion forecast) on the user interface via electronic commerce site APIs and map service APIs. Furthermore, the provision unit can link with gym reservation functions and trial application functions, enabling the user to make reservations or inquiries with a single tap. This enables users to instantly obtain gym information optimized for their health status and living area, and take concrete actions for health maintenance and improvement. As a technical effect, the provision unit achieves improved accuracy in personalized gym recommendation through automatic linkage between health condition determination results, location information, and gym databases, improved user experience, and efficiency in forming exercise habits. Specific application fields include health management apps, fitness support services, corporate health management support, and exercise guidance in nursing care settings.

The analysis unit can automatically recognize features such as complexion, swelling, puffiness, wrinkles, and scars. Complexion may include, for example, hue, brightness, saturation, and the like, but is not limited to such examples. For instance, the analysis unit analyzes the hue of the complexion to determine health status. The analysis unit can also analyze facial swelling to evaluate the degree of expansion and skin elasticity. Furthermore, the analysis unit can analyze facial puffiness to evaluate the degree of local expansion and the presence or absence of pain. The analysis unit can analyze facial wrinkles to evaluate the depth, number, and position of wrinkles. The analysis unit can also analyze facial scars to evaluate the size, depth, and position of scars. Thus, by automatically recognizing facial features, the analysis unit improves the accuracy of health status analysis. Specifically, the analysis unit receives a facial image tensor (e.g., 224×224×3 pixels) from the imaging unit as input, and performs preprocessing such as face region detection (e.g., MTCNN, YOLO-based), facial part landmark extraction (e.g., 68-point feature detection), and color space conversion (e.g., RGB→HSV). The analysis unit uses image recognition models such as convolutional neural networks (CNN) or Vision Transformers to extract feature quantities such as complexion (hue, brightness, saturation histogram), swelling (contour expansion degree, face width, cheek expansion score), puffiness (left-right asymmetry, local expansion degree), wrinkles (depth, number, position), and scars (size, depth, position by edge detection and region segmentation) as a multidimensional vector. For example, for complexion, the system outputs values such as “hue: 210 degrees, brightness: 0.72, saturation: 0.38”; for swelling, “cheek expansion degree: 0.15”; for wrinkles, “depth: 0.42, number: 8, position: corners of eyes, forehead”, etc. The analysis unit can track these feature quantities over time to detect changes in health status or abnormal signs with high accuracy. Furthermore, the analysis unit can extract fine texture features of the facial surface (e.g., GLCM, LBP features) for skin elasticity evaluation, and apply region segmentation algorithms (e.g., U-Net) for evaluation of scars and puffiness. Thus, the analysis unit realizes automatic extraction and quantification of high-dimensional features that are difficult for human visual inspection, greatly improving the accuracy, reproducibility, and speed of health status analysis. As a technical effect, the analysis unit achieves improved accuracy in health status estimation from facial images through automatic extraction of multidimensional feature quantities, earlier abnormality detection, and automation and efficiency of analysis work. Specific application fields include health management apps, remote medical support, health monitoring in nursing care settings, and skincare advice.

The determination unit can determine the correlation between the state of the face and diseases based on data accumulated in medical settings. Data accumulated in medical settings may include, for example, hospital medical records, health checkup results, genetic information, and the like, but is not limited to such examples. For instance, the determination unit determines the correlation between the state of the face and diseases based on hospital medical records. The determination unit can also determine the correlation between the state of the face and diseases based on health checkup results. Furthermore, the determination unit can determine the correlation between the state of the face and diseases based on genetic information. Thus, by making determinations based on medical data, the determination unit improves the accuracy of health status determination. Specifically, the determination unit receives a facial image feature vector (e.g., complexion score, swelling score, puffiness score, wrinkle depth, scar area size, etc., in a numerical vector of 10 or more dimensions) from the analysis unit as input. The determination unit matches these feature vectors with a large-scale structured database of paired facial images and diagnostic results accumulated in medical settings (e.g., more than 100,000 facial images and diagnostic labels, health checkup values, genetic polymorphism information, etc.). The determination unit uses a supervised learning machine learning model (e.g., multilayer perceptron, random forest, support vector machine, or deep neural network) to infer the correlation between the state of the face and disease risk. For example, the determination unit inputs complexion scores and swelling scores and outputs probability distributions such as “anemia risk: high (probability 0.81)”, “kidney disease risk: medium (probability 0.57)”, etc. The determination unit inputs health checkup results (e.g., blood test values, liver function values, kidney function values, etc.) and genetic information (e.g., SNP data, family history) as additional features to the model, and by integratively analyzing multiple data sources, further improves determination accuracy. The determination unit performs threshold determination (e.g., high risk for values above 0.7) and rule-based branching on the output disease risk probability values to determine notification content and subsequent actions for the user. During model training, the determination unit can apply cross-validation and ensemble learning to prevent overfitting and improve the robustness of determinations. Unlike conventional manual visual determination or simple rule-based determination, the determination unit realizes unconventional and highly reproducible health status estimation by integratively analyzing high-dimensional feature quantities and diverse medical data. As a technical effect, the determination unit achieves improved accuracy in health status estimation through automatic linkage between facial image feature quantities and medical databases, early detection of disease risk, immediate provision of user-optimized services, and efficiency in data management and communication load. Specific application fields include health management apps for general consumers, remote medical support, corporate health management support, risk assessment for insurance companies, and health monitoring in nursing care settings.

The imaging unit can estimate the user's emotion and adjust the imaging timing based on the estimated emotion of the user. For example, if the user is relaxed, the imaging unit performs imaging at a relaxed timing to capture a natural expression. If the user is tense, the imaging unit can provide time for relaxation before imaging to help relieve tension. Furthermore, if the user is in a hurry, the imaging unit can perform imaging quickly to avoid wasting the user's time. Thus, by adjusting the imaging timing based on the user's emotion, the imaging unit can capture natural expressions. Emotion estimation is realized, for example, by using an emotion engine or generative AI with emotion estimation functions. Generative AI may include text generation AI (e.g., LLM) or multimodal generative AI, but is not limited to such examples. Specifically, the imaging unit inputs the user's facial image tensor (e.g., 128×128×3 pixels), audio waveform data (e.g., 3 seconds of 16kHz sampled audio), and text input (e.g., user's utterances or chat history) into an emotion estimation AI model. The emotion estimation AI model uses convolutional neural networks (CNN), recurrent neural networks (RNN), Transformer-based multimodal models, etc., to output emotion labels such as “relaxed”, “tense”, “in a hurry”, and emotion scores (e.g., relaxation level 0.72, tension level 0.18). For example, facial images are used to extract features such as facial muscle movement, eye opening/closing degree, and mouth corner elevation, while audio is used to extract features such as pitch, speech rate, and intonation. The AI model integrates these features to estimate the emotional state as a multidimensional vector. The imaging unit sends signals to the imaging timing control module according to the estimated emotion scores, and, for example, automatically performs imaging when the relaxation level is 0.7 or higher, and if the tension level is high, displays guidance such as “Please take a deep breath” before delaying imaging. The imaging unit monitors the user's emotional state in real time and captures the shutter at the optimal timing to obtain natural expressions and high-quality facial images. Unlike conventional simple timer imaging or user-dependent imaging, imaging timing control linked with AI-based emotion estimation greatly improves facial image quality and analysis accuracy. As a technical effect, the imaging unit achieves improved facial image quality, user experience, and analysis accuracy through imaging timing optimization linked with emotion estimation AI. Specific application fields include health management apps, remote medical facial image acquisition, identity verification systems, and skincare advice.

The imaging unit can add a function to automatically adjust the orientation and expression of the user's face during imaging. For example, the imaging unit automatically adjusts the camera position so that the user's face faces forward. The imaging unit can also display guidance to help the user relax and achieve a natural expression. Furthermore, the imaging unit can automatically adjust the lighting so that the user's face appears bright. Thus, by automatically adjusting the orientation and expression of the user's face, the imaging unit can capture optimal facial photographs. Specifically, the imaging unit uses real-time face detection algorithms (e.g., MTCNN, MediaPipe Face Mesh) to extract facial landmarks (e.g., 68-point feature coordinates) from the camera feed. The imaging unit estimates the orientation of the face (yaw, pitch, roll angles), and if the face is not facing forward, displays guidance such as “Please turn your face a little more to the right” on the screen. The imaging unit uses expression recognition AI (e.g., CNN-based expression classification model) to determine whether the user's expression is close to “neutral”, “smile”, “frown”, etc., and provides audio or text guidance such as “Please relax” until a natural expression is obtained. For lighting adjustment, the imaging unit automatically controls the camera's exposure value and white balance, analyzes the brightness histogram of the face region, and adjusts the LED light or screen brightness to achieve optimal brightness. Furthermore, if part of the face is in shadow or backlit, the imaging unit can instruct the user to “Please adjust the position of the lighting”. These processes are executed in real time in tens of milliseconds, minimizing user burden and obtaining high-quality facial images. Unlike conventional static imaging settings or user-dependent imaging, automatic optimization of face orientation, expression, and lighting by AI greatly improves analysis accuracy and user experience. As a technical effect, the imaging unit achieves improved facial image quality, analysis accuracy, and reduced user operation burden through automatic optimization of face orientation, expression, and lighting. Specific application fields include health management apps, remote medical facial image acquisition, identity verification systems, and skincare advice.

The imaging unit can use light of different wavelengths during imaging to more accurately capture the condition of the user's skin. For example, the imaging unit uses infrared light to capture the condition of the deeper layers of the skin. The imaging unit can also use ultraviolet light to capture the condition of the skin surface in detail. Furthermore, the imaging unit can use visible light to accurately capture the color and texture of the skin. Thus, by using light of different wavelengths, the imaging unit can more accurately capture the condition of the skin. Specifically, the imaging unit is equipped with a multispectral camera module and simultaneously acquires multiple images in different wavelength bands such as visible light (wavelength 400-700 nm), near-infrared (wavelength 700-900 nm), and ultraviolet (wavelength 300-400 nm). The imaging unit analyzes blood flow status, melanin distribution, and presence of subcutaneous edema from infrared images, detects the condition of the stratum corneum, epidermal damage, spots, and freckles from ultraviolet images, and extracts skin color, texture, and pore condition with high accuracy from visible light images. The imaging unit integrates these different wavelength images into a multidimensional tensor (e.g., 224×224×5 bands) and inputs them into the analysis unit or AI model to evaluate the skin condition deeply and from multiple perspectives. For example, the system extracts feature quantities such as subcutaneous edema score 0.32 from infrared images, epidermal damage score 0.45 from ultraviolet images, and skin color score 0.78 from visible light images. The imaging unit uses a lighting control module to irradiate each wavelength at optimal intensity and angle, minimizing the effects of reflected and scattered light. Unlike conventional single-wavelength imaging or visual observation, simultaneous evaluation of deep, surface, and color tone of the skin becomes possible, greatly improving the accuracy of health condition determination and skincare advice. As a technical effect, the imaging unit achieves improved accuracy in skin condition analysis, earlier abnormality detection, and automation and efficiency of analysis work through multispectral image acquisition. Specific application fields include health management apps, skincare advice, remote medical support, and dermatology diagnostic support.

The imaging unit can estimate the user's emotion and adjust the imaging environment based on the estimated emotion of the user. For example, if the user is relaxed, the imaging unit uses soft lighting and a calm background. If the user is tense, the imaging unit can use bright lighting and a simple background. Furthermore, if the user is enjoying themselves, the imaging unit can use colorful lighting and a fun background. Thus, by adjusting the imaging environment based on the user's emotion, the imaging unit can provide the optimal imaging environment. Emotion estimation is realized, for example, by using an emotion engine or generative AI with emotion estimation functions. Generative AI may include text generation AI (e.g., LLM) or multimodal generative AI, but is not limited to such examples. Specifically, the imaging unit inputs the user's facial image tensor (e.g., 128×128×3), audio waveform data (e.g., 2 seconds of 16kHz audio), and text input (e.g., chat history) into an emotion estimation AI model. The emotion estimation AI model uses CNN or Transformer-based multimodal models to output emotion labels such as “relaxed”, “tense”, “enjoying”, and scores (e.g., relaxation level 0.68, tension level 0.21, enjoyment level 0.54). The imaging unit sends signals to the lighting control module and background switching module according to the estimated emotion scores, and, for example, if the relaxation level is high, automatically sets soft LED lighting with a color temperature of 4000 K and a calm monochrome background; if the tension level is high, selects bright lighting and a simple background; and if the enjoyment level is high, displays colorful lighting or an illustrated background by compositing. Background switching is realized using green screen compositing or real-time image processing (e.g., segmentation AI). These environment adjustments reflect the user's emotional state in real time and automatically generate the optimal imaging environment. Unlike conventional static imaging environments or user-dependent settings, environment optimization linked with AI-based emotion estimation greatly improves facial image quality and user experience. As a technical effect, the imaging unit achieves improved facial image quality, user experience, and analysis accuracy through imaging environment optimization linked with emotion estimation AI. Specific application fields include health management apps, remote medical facial image acquisition, identity verification systems, and skincare advice.

The imaging unit can automatically select imaging settings by referring to the user's past imaging data during imaging. For example, the imaging unit automatically applies lighting settings that the user preferred in the past. The imaging unit can also automatically set backgrounds previously selected by the user. Furthermore, the imaging unit can select the optimal imaging timing based on the time of day when the user previously took images. Thus, by referring to past imaging data, the imaging unit can automatically select the optimal imaging settings. Specifically, the imaging unit maintains a structured database of each user's past imaging metadata (e.g., lighting intensity, color temperature, background type, imaging time, imaging location, face orientation, expression score, etc.), and quickly searches past imaging history using the user ID as a key at the start of imaging. The imaging unit aggregates the settings parameters of the past 10 imaging sessions in chronological order, and extracts frequent values or settings highly rated by the user, such as “lighting intensity: 80%”, “color temperature: 4500 K”, “background: solid gray”, “imaging time: 8 a.m.”, etc. The imaging unit combines these history data with current environmental sensor information (e.g., ambient brightness, camera position, face detection results) and executes an algorithm (e.g., decision tree, k-NN, or reinforcement learning-based setting optimization model) to automatically determine the optimal imaging settings. The imaging unit records the types of background images or virtual backgrounds previously selected by the user, and automatically resets the background when the same or similar health or emotional state is estimated. Furthermore, the imaging unit analyzes the distribution of the user's past imaging times and automatically generates rules such as “prioritize natural light for morning imaging” or “strengthen LED lighting at night”, and controls the optimal imaging timing and lighting according to the current time. The imaging unit previews these automatic settings on the user interface, and if the user manually fine-tunes them, the operation history is accumulated as new learning data. As a technical effect, the imaging unit achieves significant improvements in facial image quality, consistency, and reproducibility, as well as reduced user operation burden and automation of the imaging process, through optimization of imaging settings using each user's past data. Unlike conventional static imaging settings or user-dependent manual adjustments, the combination of AI-based history reference and real-time optimization algorithms enables personalized imaging conditions and high-precision facial image acquisition. Specific application fields include health management apps, remote medical facial image acquisition, identity verification systems, skincare advice, and remote work profile image generation.

The imaging unit can propose an imaging location by considering the user's location information during imaging. For example, if the user is outdoors, the imaging unit proposes a location with optimal natural light. If the user is indoors, the imaging unit can propose a location with good lighting. Furthermore, if the user is on the move, the imaging unit can propose a quiet location. Thus, by considering the user's location information, the imaging unit can propose the optimal imaging location. Specifically, the imaging unit acquires the current location (latitude, longitude, altitude) using the GPS sensor of the user device or Wi-Fi/Bluetooth beacon information, and automatically determines whether the user is outdoors, indoors, or on the move using an indoor/outdoor determination algorithm (e.g., combination of surrounding Wi-Fi strength, barometric sensor, accelerometer data). If outdoors, the imaging unit estimates the intensity and direction of natural light from sun elevation and weather APIs (e.g., meteorological database linkage), and proposes specific imaging spots on the map such as “a shaded area without backlight” or “south-facing balcony in the morning”. If indoors, the imaging unit analyzes the real-time brightness histogram from the device's camera feed and guides the user to the location with the brightest and most uniform lighting (e.g., by the window, directly under a light) on the screen. If on the move, the imaging unit detects device shaking and movement speed from the accelerometer and gyroscope, and automatically generates messages such as “Please take the photo when stationary” or “We recommend taking the photo at a nearby bench or cafe”. The imaging unit ranks optimal imaging location candidates in conjunction with location and environmental information and presents them as options to the user. Furthermore, the imaging unit learns from past imaging history and user preferences (e.g., tendency to prefer outdoor imaging) to realize personalized imaging location recommendations. As a technical effect, the imaging unit achieves significant improvements in facial image quality, analysis accuracy, and user experience through optimization of imaging location by integrating location information, environmental sensors, and past history. Unlike conventional user-dependent imaging location selection or static guidance, real-time environmental analysis and personalized recommendation by AI enable automatic proposals of optimal imaging conditions. Specific application fields include health management apps, remote medical facial image acquisition, identity verification systems, skincare advice, and remote work profile image generation.

The analysis unit can estimate the user's emotion and adjust the accuracy of analysis based on the estimated emotion of the user. For example, if the user is relaxed, the analysis unit performs detailed analysis. If the user is tense, the analysis unit can perform simplified analysis. Furthermore, if the user is in a hurry, the analysis unit can perform rapid analysis. Thus, by adjusting the accuracy of analysis based on the user's emotion, the analysis unit can provide more accurate analysis results. Emotion estimation is realized, for example, by using an emotion engine or generative AI with emotion estimation functions. Generative AI may include text generation AI (e.g., LLM) or multimodal generative AI, but is not limited to such examples. Specifically, the analysis unit inputs the user's facial image tensor (e.g., 128×128×3 pixels), audio waveform data (e.g., 2 seconds of 16 kHz audio), and text input (e.g., chat history or utterances) into an emotion estimation AI model. The emotion estimation AI model uses CNN or Transformer-based multimodal models to output emotion labels such as “relaxed”, “tense”, “in a hurry”, and scores (e.g., relaxation level 0.68, tension level 0.21, hurry level 0.54). The analysis unit dynamically switches the detail level of the image analysis pipeline according to the estimated emotion scores. For example, if the relaxation level is high, the system uses high-resolution images (e.g., 256×256×3), multi-stage feature extraction (e.g., all features of complexion, swelling, puffiness, wrinkles, scars), time-series change analysis, and skin texture analysis (e.g., GLCM, LBP). If the tension level is high, the system limits to low-resolution images (e.g., 128×128×3) and only major features (e.g., complexion, swelling), shortening processing time. If the hurry level is high, the system uses minimal feature extraction and fast inference mode (e.g., lightweight CNN model) to output analysis results immediately. The analysis unit performs threshold determination on the output values of the emotion estimation AI and automatically adjusts analysis accuracy, processing load, and output content to provide analysis services optimized for the user's situation. As a technical effect, the analysis unit achieves improved user experience, efficient use of computational resources, and optimal balance between analysis accuracy and response speed through analysis accuracy optimization linked with emotion estimation AI. Specific application fields include health management apps, remote medical support, health monitoring in nursing care settings, and skincare advice.

The analysis unit can perform high-resolution image analysis to detect subtle changes in the face during analysis. For example, the analysis unit uses a high-resolution camera to capture subtle changes in the face. The analysis unit can also optimize image analysis algorithms to detect subtle changes. Furthermore, the analysis unit can combine multiple images to analyze subtle changes in the face. Thus, by performing high-resolution image analysis, the analysis unit can accurately detect subtle changes in the face. Specifically, the analysis unit receives facial image tensors obtained from a high-resolution camera module (e.g., resolution of 1920×1080 pixels or higher) as input, and performs preprocessing such as face region detection, facial part landmark extraction (e.g., 68-point feature detection), and noise removal (e.g., bilateral filter). The analysis unit uses high-resolution compatible image recognition models such as convolutional neural networks (CNN) or Vision Transformers to extract fine texture features of the facial surface (e.g., wrinkle depth in 0.12 mm units, pore diameter in 0.05 mm units, minute skin color irregularities, and visible blood vessels) as a multidimensional vector. The analysis unit compares high-resolution images at multiple time points in chronological order, generates difference images, and extracts dynamic features (e.g., minute movements of facial muscles, changes in skin firmness and sagging). Furthermore, the analysis unit optimizes image analysis algorithms (e.g., multi-scale tensor analysis, wavelet transform, region segmentation AI) to detect subtle changes with high sensitivity and low error. The analysis unit utilizes detected subtle changes for subsequent processing such as health status estimation, skincare advice, and disease risk assessment. As a technical effect, the analysis unit achieves significant improvements in automatic detection accuracy, reproducibility, and analysis speed of subtle changes in the face through the combination of high-resolution image analysis and optimized algorithms. Unlike conventional low-resolution images or visual observation, high-precision health status analysis and abnormality detection become possible. Specific application fields include health management apps, remote medical support, skincare advice, and dermatology diagnostic support.

The analysis unit can refer to the user's past health data during analysis to improve the accuracy of the analysis result. For example, the analysis unit refers to the user's past health checkup results to supplement the analysis result. The analysis unit can also refer to the user's past medical history to supplement the analysis result. Furthermore, the analysis unit can improve the reliability of the analysis result based on the user's past health data. Thus, by referring to past health data, the analysis unit improves the accuracy of the analysis result. Specifically, the analysis unit maintains a structured database for each user, including health checkup results (e.g., blood test values, liver and kidney function values, weight and BMI trends), medical history (e.g., past illnesses, surgical history, medication history), and past facial image analysis results (e.g., time-series data of complexion, swelling, wrinkle scores). The analysis unit integrates the latest feature vector extracted from facial images (e.g., complexion score 0.72, swelling score 0.18, etc.) with past health data and inputs them into a machine learning model (e.g., multilayer perceptron, random forest, time-series analysis model) to perform correction and reliability evaluation of the analysis result. For example, for users with a history of anemia, the system detects even slight paleness in complexion with high sensitivity, and for users with a history of kidney disease, individually optimizes the threshold for swelling scores. The analysis unit uses Bayesian estimation and personalized weighting based on past health data to calculate a reliability index for the analysis result (e.g., reliability 0.92) and transmits it to subsequent determination and provision units. As a technical effect, the analysis unit achieves improved accuracy and reliability of analysis, reduced risk of misjudgment, and realization of personalized health management by utilizing each user's individual health history. Unlike conventional single-point data analysis or uniform determination, time-series and individual history reference by AI enables detailed health status analysis. Specific application fields include health management apps, remote medical support, risk assessment for insurance companies, and health monitoring in nursing care settings.

The analysis unit can estimate the user's emotion and adjust the display method of the analysis result based on the estimated emotion of the user. For example, if the user is relaxed, the analysis unit displays detailed analysis results. If the user is tense, the analysis unit can display simplified analysis results. Furthermore, if the user is in a hurry, the analysis unit can display analysis results that focus on key points. Thus, by adjusting the display method of the analysis result based on the user's emotion, the analysis unit can provide a display that is easy for the user to understand. Emotion estimation is realized, for example, by using an emotion engine or generative AI with emotion estimation functions. Generative AI may include text generation AI (e.g., LLM) or multimodal generative AI, but is not limited to such examples. Specifically, the analysis unit inputs the user's facial image tensor (e.g., 128×128×3 pixels), audio waveform data (e.g., 2 seconds of 16 kHz audio), and text input (e.g., chat history or utterances) into an emotion estimation AI model. The emotion estimation AI model uses convolutional neural networks or Transformer-based multimodal models to output emotion labels such as “relaxed”, “tense”, “in a hurry”, and emotion scores (e.g., relaxation level 0.68, tension level 0.21, hurry level 0.54). For example, facial images are used to extract features such as facial muscle movement, eye opening/closing degree, and mouth corner elevation, while audio is used to extract features such as pitch, speech rate, and intonation. The AI model integrates these features to estimate the emotional state as a multidimensional vector. The analysis unit automatically switches the display mode of the analysis result according to the estimated emotion scores. For example, if the relaxation level is high, the system displays detailed analysis results including all features of complexion, swelling, puffiness, wrinkles, scars, time-series change graphs, reliability indices, and comparisons with past data. If the tension level is high, the system displays only major health indicators (e.g., complexion score, swelling score, overall health level) as concise text and icons, and adds explanations of technical terms and reassuring comments. If the hurry level is high, the system immediately presents only the key points (e.g., “Health status: good”, “Notes: none”) in large font and color coding. The analysis unit dynamically adjusts the detail level, layout, color scheme, and font size of the display content, and automatically generates an interface optimized for the user's emotional state. Furthermore, the analysis unit learns from the user's past display history and feedback to evolve individually optimized display patterns. As a technical effect, the analysis unit achieves improved user experience, information comprehension, stress reduction, and acceptance of analysis results through display optimization of analysis results linked with emotion estimation AI. Unlike conventional uniform result display or user-dependent display switching, real-time emotion estimation and automatic display optimization by AI enable optimal information presentation for each user. Specific application fields include health management apps, remote medical support, health monitoring in nursing care settings, skincare advice, and identity verification systems.

The analysis unit can refer to the user's lifestyle data during analysis to supplement the analysis result. For example, the analysis unit refers to the user's dietary data to supplement the analysis result. The analysis unit can also refer to the user's exercise habit data to supplement the analysis result. Furthermore, the analysis unit can refer to the user's sleep data to supplement the analysis result. Thus, by referring to lifestyle data, the analysis unit improves the reliability of the analysis result. Specifically, the analysis unit maintains a structured database for each user, including dietary records (e.g., calorie intake, nutrient balance, number of meals, meal times), exercise records (e.g., step count, calories burned, exercise type, exercise frequency), and sleep records (e.g., bedtime and wake-up time, sleep duration, sleep efficiency, proportion of deep sleep). The analysis unit integrates the latest health feature vector extracted from facial images (e.g., complexion score, swelling score, etc.) with lifestyle data and inputs them into a machine learning model (e.g., multilayer perceptron, random forest, time-series analysis model) to perform correction and reliability evaluation of the analysis result. For example, if the dietary data shows a tendency for low vegetable intake or frequent high-fat meals, the system detects changes in complexion or skin condition with high sensitivity; if exercise habits are lacking, the system evaluates the risk of swelling or puffiness more highly. If sleep data is insufficient, the system weights features such as dark circles under the eyes or dull complexion. The analysis unit uses Bayesian estimation and personalized weighting based on lifestyle data to calculate a reliability index for the analysis result (e.g., reliability 0.91) and transmits it to subsequent determination and provision units. Furthermore, the analysis unit analyzes time-series changes in lifestyle data to detect trends of health deterioration or improvement at an early stage. As a technical effect, the analysis unit achieves improved accuracy and reliability of analysis, reduced risk of misjudgment, and realization of personalized health management by utilizing each user's individual lifestyle data. Unlike conventional single-point data analysis or uniform determination, multidimensional and time-series data integration by AI enables detailed health status analysis. Specific application fields include health management apps, remote medical support, risk assessment for insurance companies, health monitoring in nursing care settings, and corporate health management support.

The analysis unit can improve the accuracy of the analysis result by considering the user's genetic information during analysis. For example, the analysis unit analyzes disease risk based on the user's genetic information. The analysis unit can also analyze health status based on the user's genetic information. Furthermore, the analysis unit can improve the reliability of the analysis result based on the user's genetic information. Thus, by considering genetic information, the analysis unit improves the accuracy of the analysis result. Specifically, the analysis unit maintains a structured database for each user, including genetic polymorphism information (e.g., SNP data, presence of disease-related genes, family history). The analysis unit integrates the health feature vector extracted from facial images (e.g., complexion score, swelling score, etc.) with genetic information and inputs them into a machine learning model (e.g., multilayer perceptron, random forest, gene ×phenotype integration model) to improve the accuracy of disease risk estimation and health status analysis. For example, if a specific genetic polymorphism is related to anemia risk or kidney disease risk, the system individually optimizes the determination threshold for features such as complexion and swelling. The analysis unit uses Bayesian estimation and personalized weighting based on genetic information to calculate a reliability index for the analysis result (e.g., reliability 0.95) and transmits it to subsequent determination and provision units. Furthermore, the analysis unit integratively analyzes genetic information with lifestyle data, health checkup results, and other multidimensional data to generate comprehensive health risk assessments and preventive advice. As a technical effect, the analysis unit achieves improved accuracy and reliability of analysis, reduced risk of misjudgment, and realization of personalized health management by utilizing each user's individual genetic information. Unlike conventional uniform determination or single data source analysis, integrated analysis of genetic information by AI enables detailed health status analysis and disease risk prediction. Specific application fields include health management apps, remote medical support, risk assessment for insurance companies, health monitoring in nursing care settings, and genetic counseling support.

The determination unit can estimate the user's emotion and adjust the reliability of the determination result based on the estimated emotion of the user. For example, if the user is relaxed, the determination unit provides detailed determination results. If the user is tense, the determination unit can provide simplified determination results. Furthermore, if the user is in a hurry, the determination unit can provide rapid determination results. Thus, by adjusting the reliability of the determination result based on the user's emotion, the determination unit can provide more accurate determination results. Emotion estimation is realized, for example, by using an emotion engine or generative AI with emotion estimation functions. Generative AI may include text generation AI (e.g., LLM) or multimodal generative AI, but is not limited to such examples. Specifically, the determination unit receives the facial image feature vector (e.g., complexion score, swelling score, etc.) from the analysis unit and the output of the emotion estimation AI model (e.g., relaxation level 0.72, tension level 0.18, hurry level 0.54) as input. The determination unit dynamically switches the detail level of the determination algorithm and the display method of reliability indices according to the emotion scores. For example, if the relaxation level is high, the system presents detailed probability distributions of disease risk (e.g., “anemia risk: high (probability 0.81)”), underlying feature quantities, comparisons with past data, reliability indices (e.g., reliability 0.93), etc. If the tension level is high, the system displays only major determination results (e.g., “Health status: good”, “Note: tendency for swelling”) as concise text and icons, and adds reassuring comments. If the hurry level is high, the system immediately presents only the key points (e.g., “No abnormality”, “Caution required”) and omits detailed explanations. The determination unit performs threshold determination on the output values of the emotion estimation AI and automatically adjusts the detail level, reliability indices, and display content of the determination result to provide determination services optimized for the user's situation. Furthermore, the determination unit learns from the user's past feedback and display history to evolve individually optimized determination patterns. As a technical effect, the determination unit achieves improved user experience, information comprehension, stress reduction, and acceptance of determination results through reliability optimization of determination results linked with emotion estimation AI. Unlike conventional uniform determination result display or user-dependent switching, real-time emotion estimation and automatic reliability adjustment by AI enable optimal determination information presentation for each user. Specific application fields include health management apps, remote medical support, health monitoring in nursing care settings, and risk assessment for insurance companies.

The determination unit can refer to multiple medical databases during determination to improve the accuracy of the determination result. For example, the determination unit may integrate multiple medical databases to supplement the determination result. Additionally, the determination unit can refer to the latest information in medical databases to update the determination result. Furthermore, the determination unit can evaluate the reliability of medical databases to further improve the accuracy of the determination result. Thus, by referring to multiple medical databases, the determination unit enhances the accuracy of the determination result. Specifically, the determination unit collaborates via API with multiple medical databases provided by domestic and international medical institutions and research organizations (e.g., medical record databases, health checkup databases, genetic polymorphism databases, disease risk statistics databases), and matches facial image feature vectors and health checkup values. The determination unit integrates diagnostic labels, risk scores, and statistical values obtained from each database, and uses ensemble learning models (e.g., random forest, gradient boosting, stacking) or Bayesian estimation models to generate determination results by weighted averaging and reliability evaluation of information from multiple data sources. For example, if a domestic database yields “anemia risk: high (probability 0.78)” and an overseas database yields “anemia risk: medium (probability 0.62)”, the final determination is corrected by considering reliability and recency. The determination unit automatically evaluates meta-information such as update date, data volume, and source reliability of each database, and preferentially utilizes highly reliable data. Furthermore, the determination unit periodically acquires the latest information from medical databases (e.g., new disease risk factors, revised treatment guidelines) and automatically updates determination algorithms and thresholds. The technical effects are that the determination unit achieves improved accuracy and reliability of determinations by integrated reference to multiple medical databases, immediate reflection of the latest medical knowledge, and reduction of misdiagnosis risk. Unlike conventional approaches that depend on a single database or static determination logic, high-precision and up-to-date health status determination is enabled by AI-based multidimensional and dynamic data integration. Specific application fields include health management apps, remote medical support, risk assessment for insurance companies, health monitoring in nursing care settings, and medical research support.

The determination unit can refer to the user's past health checkup results during determination to supplement the determination result. For example, the determination unit may supplement the determination result based on the user's past health checkup results. Additionally, the determination unit can refer to the user's past health checkup results to improve the reliability of the determination result. Furthermore, the determination unit can determine disease risk based on the user's past health checkup results. Thus, by referring to past health checkup results, the determination unit enhances the reliability of the determination result. Specifically, the determination unit maintains health checkup results for each user (e.g., blood test values, liver and kidney function values, weight and BMI trends) as a structured database, and integrates facial image feature vectors and the latest health indicators with past health checkup data as input to machine learning models (e.g., multilayer perceptron, random forest, time series analysis models). The determination unit calculates reliability indicators for the determination result (e.g., reliability 0.92) using Bayesian estimation and personalized weighting based on past health checkup results, and strengthens risk determination if abnormal values or risk trends persist. For example, for users with a history of anemia, even slight paleness in complexion is detected with high sensitivity, and for those with a history of kidney disease, the threshold for swelling scores is individually optimized. The determination unit analyzes time-series changes in health checkup results to detect early signs of health deterioration or improvement. The technical effects are that the determination unit achieves improved accuracy and reliability of determinations, reduced risk of misdiagnosis, and realization of personalized health management by utilizing individual health checkup histories. Unlike conventional single-point data determination or uniform determinations, fine-grained health status determination is enabled by AI-based time-series and individual history reference. Specific application fields include health management apps, remote medical support, risk assessment for insurance companies, and health monitoring in nursing care settings.

The determination unit can estimate the user's emotion and adjust the notification method of the determination result based on the estimated emotion of the user. For example, if the user is relaxed, the determination unit provides detailed notifications. If the user is tense, the determination unit can provide simplified notifications. Furthermore, if the user is in a hurry, the determination unit can provide rapid notifications. Thus, by adjusting the notification method based on the user's emotion, the determination unit can provide optimal notifications for the user. Emotion estimation is realized using, for example, an emotion engine or generative AI with emotion estimation functions. Generative AI may include text generation AI (e.g., LLM) or multimodal generative AI, but is not limited to these examples. Specifically, the determination unit receives as input the facial image feature vector (e.g., complexion score, swelling score, puffiness score, etc., as a numerical vector of 10 or more dimensions) from the analysis unit and the output of the emotion estimation AI model (e.g., relaxation level 0.72, tension level 0.18, hurry level 0.54). The emotion estimation AI model takes as input facial image tensors (e.g., 128×128×3 pixels), audio waveform data (e.g., 2 seconds of 16 kHz audio), and text input (e.g., chat history or spoken content), and uses convolutional neural networks or Transformer-based multimodal models to output emotion labels and scores such as “relaxed”, “tense”, or “in a hurry” from image, audio, and text. For example, facial images are used to extract features such as facial muscle movement, eye opening/closing degree, and mouth corner elevation, while audio is used to extract features such as pitch, speech rate, and intonation. The AI model integrates these features to estimate the emotional state as a multidimensional vector. According to the estimated emotion scores, the determination unit sends signals to the notification method control module: if relaxation level is high, a detailed notification (e.g., detailed report including determination rationale, comparison with past data, reliability indicators) is displayed on the user interface; if tension level is high, only the main determination results (e.g., “Health status: Good”, “Caution: Swelling tendency”) are notified as concise text and icons; if hurry level is high, only the key points (e.g., “No abnormality”, “Caution required”) are immediately notified as pop-ups. The determination unit dynamically adjusts the detail level, layout, color scheme, and font size of the notification content, and automatically generates a notification interface optimized for the user's emotional state. Furthermore, the determination unit can learn from the user's past notification history and feedback to evolve individually optimized notification patterns. Notification timing and means (e.g., in-app notification, email, push notification) are also automatically selected according to emotional state and user usage trends. The technical effects are that the determination unit achieves improved user experience, information comprehension, stress reduction, and acceptance of determination results by optimizing notification methods in conjunction with emotion estimation AI. Unlike conventional uniform notifications or user-dependent notification settings, optimal information delivery for each user is enabled by AI-based real-time emotion estimation and automatic notification optimization. Specific application fields include health management apps, remote medical support, health monitoring in nursing care settings, risk assessment for insurance companies, and identity verification systems.

The determination unit can refer to the user's family health data during determination to supplement the determination result. For example, the determination unit may supplement the determination result based on the user's family health data. Additionally, the determination unit can refer to the user's family health data to improve the reliability of the determination result. Furthermore, the determination unit can determine disease risk based on the user's family health data. Thus, by referring to family health data, the determination unit enhances the reliability of the determination result. Specifically, the determination unit maintains, for each user, family health checkup results (e.g., blood test values, medical history, hereditary disease risk, presence of lifestyle diseases), family facial image analysis results (e.g., time-series data of complexion, swelling, wrinkle scores), and family lifestyle data (e.g., diet, exercise, sleep records) as a structured database. The determination unit integrates the user's facial image feature vector and health checkup values with family health data, and inputs them into machine learning models (e.g., multilayer perceptron, random forest, family risk estimation models) to improve the accuracy of disease risk estimation and health status determination considering family history. For example, if there is a family history of hypertension or diabetes, even slight abnormalities in the user's blood pressure or blood glucose are detected with high sensitivity; if there is a specific hereditary disease risk in the family, thresholds for features such as complexion and swelling are individually optimized. The determination unit calculates reliability indicators for the determination result (e.g., reliability 0.94) using Bayesian estimation and personalized weighting based on family data, and strengthens risk determination if abnormal trends persist within the family. Furthermore, the determination unit analyzes time-series changes in family health data to generate preventive advice reflecting disease onset trends and lifestyle influences within the family. The technical effects are that the determination unit achieves improved accuracy and reliability of determinations, early detection of hereditary disease risks, and realization of personalized health management by utilizing family health data. Unlike conventional individual-only data determination or uniform risk assessment, fine-grained health status determination and preventive medical support are enabled by AI-based integrated analysis of family data. Specific application fields include health management apps, remote medical support, risk assessment for insurance companies, health monitoring in nursing care settings, and genetic counseling support.

The determination unit can consider the user's living environment data during determination to improve the accuracy of the determination result. For example, the determination unit may supplement the determination result based on the user's living environment data. Additionally, the determination unit can refer to the user's living environment data to improve the reliability of the determination result. Furthermore, the determination unit can determine disease risk based on the user's living environment data. Thus, by considering living environment data, the determination unit enhances the accuracy of the determination result. Specifically, the determination unit maintains, for each user, living environment data (e.g., temperature, humidity, air pollution level, pollen count, noise level, water quality, age of residence, number of cohabitants, presence of pets) as a structured database. The determination unit integrates facial image feature vectors and health checkup values with living environment data, and inputs them into machine learning models (e.g., multilayer perceptron, random forest, environmental risk estimation models) to improve the accuracy of disease risk estimation and health status determination considering environmental factors. For example, if the user lives in an area with high air pollution, respiratory disease risk is evaluated as high; during periods of high pollen count, allergy risk is emphasized. If there are large fluctuations in humidity or temperature, thresholds for features such as skin dryness and swelling are individually optimized. The determination unit calculates reliability indicators for the determination result (e.g., reliability 0.93) using Bayesian estimation and personalized weighting based on living environment data, and generates preventive advice and alerts reflecting health status fluctuations due to environmental factors. Furthermore, the determination unit analyzes time-series changes in living environment data to detect early signs of seasonal disease risk or health deterioration due to environmental changes. The technical effects are that the determination unit achieves improved accuracy and reliability of determinations, early detection of environmental risk factors, and realization of personalized health management by utilizing living environment data. Unlike conventional determinations using only individual data or uniform risk assessment, fine-grained health status determination and preventive medical support are enabled by AI-based integrated analysis of environmental data. Specific application fields include health management apps, remote medical support, risk assessment for insurance companies, health monitoring in nursing care settings, and environmental medicine research support.

The provision unit can estimate the user's emotion and adjust the content of the information provided based on the estimated emotion of the user. For example, if the user is relaxed, the provision unit provides detailed information. If the user is tense, the provision unit can provide simplified information. Furthermore, if the user is in a hurry, the provision unit can provide rapid information. Thus, by adjusting the content of the information based on the user's emotion, the provision unit can provide optimal information for the user. Emotion estimation is realized using, for example, an emotion engine or generative AI with emotion estimation functions. Generative AI may include text generation AI (e.g., LLM) or multimodal generative AI, but is not limited to these examples. Specifically, the provision unit receives as input the health status determination result from the determination unit (e.g., labels and probability values such as “anemia risk: high”, “kidney disease risk: medium”) and the output of the emotion estimation AI model (e.g., relaxation level 0.68, tension level 0.21, hurry level 0.54). The emotion estimation AI model uses facial image tensors, audio waveform data, and text input, and outputs emotion labels and scores using CNN or Transformer-based multimodal models. According to the estimated emotion scores, the provision unit sends signals to the information generation module: if relaxation level is high, a detailed health report (e.g., determination rationale, comparison with past data, reliability indicators, recommended actions) is generated; if tension level is high, only the main health indicators (e.g., complexion score, swelling score, overall health level) are presented as concise text and icons; if hurry level is high, only the key points (e.g., “Health status: Good”, “No special notes”) are displayed immediately. The provision unit dynamically adjusts the detail level, layout, color scheme, and font size of the information content, and automatically generates an interface optimized for the user's emotional state. Furthermore, the provision unit can learn from the user's past information browsing history and feedback to evolve individually optimized information presentation patterns. The technical effects are that the provision unit achieves improved user experience, information comprehension, stress reduction, and promotion of healthy behavior by optimizing information content in conjunction with emotion estimation AI. Unlike conventional uniform information presentation or user-dependent display switching, optimal information provision for each user is enabled by AI-based real-time emotion estimation and automatic information optimization. Specific application fields include health management apps, remote medical support, health monitoring in nursing care settings, skincare advice, and identity verification systems.

The provision unit can refer to the user's past behavioral history during provision to provide optimal information. For example, the provision unit may provide optimal information based on the user's past behavioral history. Additionally, the provision unit can refer to the user's past behavioral history to improve the reliability of the information. Furthermore, the provision unit can determine the priority of information based on the user's past behavioral history. Thus, by referring to past behavioral history, the provision unit can provide optimal information. Specifically, the provision unit maintains, for each user, past behavioral history data (e.g., health recipe browsing history, gym search history, health advice reception history, action execution history, information evaluation and feedback) as a structured database. The provision unit integrates health status determination results and current health indicators received from the determination unit with past behavioral history, and inputs them into machine learning models (e.g., collaborative filtering, content-based recommendation, reinforcement learning-based information optimization models) to recommend information and prioritize it reflecting the user's preferences and behavioral tendencies. For example, users who frequently browse health recipes are preferentially presented with new recipe information, and those with many gym searches are emphasized with exercise programs and nearby gym information. Information reliability is calculated by weighting the user's past evaluations and feedback, and a reliability indicator (e.g., reliability 0.91) is assigned. Furthermore, the provision unit analyzes time-series changes in behavioral history to generate personalized information reflecting continuity and improvement trends in health behavior. The technical effects are that the provision unit achieves improved relevance, reliability, and acceptance of information, promotion of healthy behavior, and improved user experience by optimizing information using individual behavioral history. Unlike conventional static information provision or uniform recommendations, personalized information provision is enabled by combining AI-based history reference and real-time optimization algorithms. Specific application fields include health management apps, remote medical support, health monitoring in nursing care settings, skincare advice, and health support for remote work.

The provision unit can provide customized advice based on the user's current health condition during provision. For example, the provision unit may provide optimal advice based on the user's current health condition. Additionally, the provision unit can refer to the user's current health condition to improve the reliability of the advice. Furthermore, the provision unit can determine the priority of advice based on the user's current health condition. Thus, by providing customized advice based on the current health condition, the provision unit can provide optimal advice for the user. Specifically, the provision unit receives as input the health status determination result from the determination unit (e.g., labels and probability values such as “anemia risk: high”, “kidney disease risk: medium”) and collaborates with a health advice database (e.g., structured data including advice ID, recommended actions, rationale, reliability, priority). The provision unit executes SQL queries or vector searches according to the determination result, and extracts advice candidates under conditions such as “recommend iron intake”, “salt restriction”, “improvement of exercise habits”. The provision unit ranks the extracted advice candidates by considering the user's past health data and behavioral history, using a recommendation engine (e.g., collaborative filtering, content-based recommendation, or Transformer-based advice recommendation model). The provision unit automatically generates and presents detailed information of the finally selected advice (e.g., recommended reason, execution procedure, expected effect, reliability indicator) on the user interface, with images or videos. Advice reliability is calculated by weighting reliability indicators from the determination unit and rationale information from the advice database. Furthermore, the provision unit dynamically adjusts the priority of advice according to the user's health condition and behavioral tendencies, and highlights the most important actions. The technical effects are that the provision unit achieves improved accuracy of personalized advice provision, improved user experience, and promotion of healthy behavior by automatic collaboration between health status determination results and the advice database. Unlike conventional uniform advice provision or user-dependent information search, optimal health support for each user is enabled by AI-based real-time health status analysis and automatic advice optimization. Specific application fields include health management apps, remote medical support, health monitoring in nursing care settings, and corporate health management support.

The provision unit can estimate the user's emotion and determine the priority of the information provided based on the estimated emotion of the user. For example, if the user is relaxed, the provision unit prioritizes detailed information. If the user is tense, the provision unit can prioritize simplified information. Furthermore, if the user is in a hurry, the provision unit can prioritize rapid information. Thus, by determining the priority of information based on the user's emotion, the provision unit can provide optimal information for the user. Emotion estimation is realized using, for example, an emotion engine or generative AI with emotion estimation functions. Generative AI may include text generation AI (e.g., LLM) or multimodal generative AI, but is not limited to these examples. Specifically, the provision unit receives as input the health status determination result from the determination unit and the output of the emotion estimation AI model (e.g., relaxation level 0.68, tension level 0.21, hurry level 0.54). The emotion estimation AI model uses facial image tensors, audio waveform data, and text input, and outputs emotion labels and scores using CNN or Transformer-based multimodal models. According to the estimated emotion scores, the provision unit sends signals to the information priority determination module: if relaxation level is high, detailed health information and recommended actions are preferentially displayed; if tension level is high, main health indicators and reassuring information are prioritized; if hurry level is high, only the key points are presented immediately. Information priority is dynamically adjusted by also considering the user's past browsing history and feedback. Furthermore, the provision unit automatically selects the timing and means of information presentation (e.g., in-app notification, push notification, email) according to emotional state and usage trends. The technical effects are that the provision unit achieves improved user experience, information comprehension, stress reduction, and promotion of healthy behavior by optimizing information priority in conjunction with emotion estimation AI. Unlike conventional uniform information presentation or user-dependent display switching, optimal information provision for each user is enabled by AI-based real-time emotion estimation and automatic priority optimization. Specific application fields include health management apps, remote medical support, health monitoring in nursing care settings, skincare advice, and identity verification systems.

The provision unit can propose optimal services by considering the user's geographic location information during provision. For example, the provision unit may propose optimal services based on the user's current location. Additionally, the provision unit can refer to the user's location information to propose nearby medical institutions. Furthermore, the provision unit can propose nearby health-related facilities based on the user's location information. Thus, by considering geographic location information, the provision unit can propose optimal services. Specifically, the provision unit acquires the current location (latitude, longitude, altitude) using the GPS sensor of the user terminal or Wi-Fi/Bluetooth beacon information, and combines it with the health status determination result received from the determination unit to collaborate with electronic commerce sites and medical institution/health facility databases (e.g., structured data including facility ID, location information, medical departments, equipment, business hours, congestion status). The provision unit uses spatial search algorithms (e.g., KD-Tree, Ball Tree) to extract facility candidates within a certain distance (e.g., within a 2 km radius) from the user's current location, and filters and ranks them according to health status and needs. For example, if anemia risk is high, medical institutions with internal medicine or hematology departments are prioritized; if exercise deficiency risk is high, gyms or fitness facilities are preferentially proposed. The provision unit automatically generates and presents detailed information of the finally selected facility (e.g., name, address, map link, equipment photos, price list, business hours, congestion forecast) on the user interface, and collaborates with map service APIs and reservation APIs. Furthermore, the provision unit learns from the user's past facility usage history and preferences to realize personalized facility recommendations. The technical effects are that the provision unit achieves improved accuracy of personalized service recommendations, improved user experience, and promotion of healthy behavior by automatic collaboration of location information, health status, and facility databases. Unlike conventional static facility search or user-dependent selection, optimal service proposals are enabled by AI-based real-time environment analysis and personalized recommendation. Specific application fields include health management apps, remote medical support, health monitoring in nursing care settings, skincare advice, and health support for remote work.

The provision unit can analyze the user's social media activity during provision to provide relevant information. For example, the provision unit may analyze the user's social media posts to provide relevant health information. Additionally, the provision unit can refer to the activities of the user's social media friends to propose related services. Furthermore, the provision unit can provide optimal information based on the user's social media interests. Thus, by analyzing social media activity, the provision unit can provide relevant information. Specifically, the provision unit acquires the user's social media post data (e.g., text, images, videos, post date/time, hashtags, like/comment history) and friend network information (e.g., friends' health-related posts, event participation history) via API, and uses natural language processing models (e.g., BERT, Transformer-based text classification models) and image recognition models (e.g., CNN, Vision Transformer) to automatically extract health-related topics and interests. For example, keywords such as “diet”, “muscle training”, “sleep improvement” or image content are used to identify the user's areas of interest, and relevant health information or services (e.g., diet recipes, muscle training videos, sleep improvement advice) are preferentially recommended. By analyzing friends' activity data, group-oriented services can be proposed if the user participates in the same event or health challenge. Furthermore, the provision unit integrates the user's past social media activity with health status determination results, and uses personalized information recommendation models (e.g., collaborative filtering, content-based recommendation) to rank optimal information. The technical effects are that the provision unit achieves improved relevance and acceptance of information, improved user experience, and promotion of healthy behavior by automatic collaboration of social media activity analysis and health information recommendation. Unlike conventional static information provision or user-dependent search, optimal health support for each user is enabled by AI-based real-time SNS analysis and automatic information optimization. Specific application fields include health management apps, remote medical support, health monitoring in nursing care settings, skincare advice, and health support for remote work.

The system according to the embodiment is not limited to the examples described above, and various modifications are possible, for example, as follows. Specifically, the system can flexibly change the module configuration for facial image analysis, health status determination, and information provision, as well as the architecture of AI models. For example, in the facial image analysis unit, not only convolutional neural networks (CNN) but also advanced image recognition models such as Vision Transformer and graph neural networks can be adopted. In the health status determination unit, ensemble learning combining various machine learning models such as random forest, support vector machine, and deep neural networks, or Bayesian estimation models can be introduced. In the information provision unit, collaborative filtering, content-based recommendation, and reinforcement learning-based recommendation engines can be utilized to realize personalized information provision according to user preferences and behavioral history. Furthermore, in addition to facial images, audio data and biometric sensor data (e.g., heart rate, blood pressure, skin temperature), as well as time-series data from wearable devices, can be input to AI models to perform multimodal health status estimation, anomaly detection, and time-series change analysis. Data flow and inter-module collaboration can also be API-based for extensibility, and variations such as cloud distributed processing, edge AI implementation, and enhanced privacy protection (e.g., federated learning, anonymization processing) can be implemented. The technical effects are that the system achieves optimization of analysis accuracy, processing speed, user experience, and security through diverse variations in AI models, data flow, and information provision methods. Specific application fields include health management apps, remote medical support, risk assessment for insurance companies, health monitoring in nursing care settings, skincare advice, health support for remote work, and corporate health management support.

The analysis unit can track changes in facial expressions in real time when analyzing a user's facial photograph and detect changes in health status. For example, the analysis unit analyzes muscle movements when the user changes from a smile to a neutral expression to estimate stress level. It can also analyze the frequency of squinting to assess fatigue level. Furthermore, the analysis unit can analyze the action of frowning to detect the presence of pain or discomfort. Thus, by tracking changes in facial expressions in real time, the analysis unit enables more detailed analysis of health status. Specifically, the analysis unit receives continuous facial image tensors (e.g., 30 frames per second, each frame 224×224×3 pixels) from the imaging unit as time-series data, and performs preprocessing such as face region detection (e.g., MTCNN), facial part landmark extraction (e.g., 68-point feature detection), and time-series vectorization of dynamic features for each facial part (e.g., mouth corner position, inter-brow distance, eye opening/closing degree). The analysis unit uses time-series analysis models such as recurrent neural networks (RNN), time-series Transformer, or 3D convolutional neural networks (3D-CNN) to automatically extract expression change patterns (e.g., smile to neutral, squinting frequency, frowning actions). Example inputs to the AI model include (1) continuous facial image tensors for 30 frames, (2) time-series vectors of facial part coordinates for each frame, and (3) time-series arrays of facial muscle activity. The AI model generates output labels and scores such as “stress level: high (score 0.74)”, “fatigue level: medium (score 0.52)”, “pain indication: present (probability 0.61)” from these inputs. For example, if the transition speed from smile to neutral is fast or squinting frequency exceeds 10 times per minute, fatigue level is determined to be high; if continuous frowning is observed, the possibility of pain or discomfort is highly evaluated. The analysis unit applies threshold determination to these output values and transmits information on abnormal signs, stress, fatigue, or pain in health status to subsequent determination and provision units. Furthermore, the analysis unit compares time-series patterns of expression changes with past user data to realize individually optimized health status estimation and anomaly detection. Unlike conventional static facial image analysis or single-frame determination, this analysis unit enables real-time and high-precision detection of health status changes through automatic extraction of high-dimensional time-series features and AI-based dynamic analysis. The technical effects are that the analysis unit achieves early detection of stress, fatigue, pain, etc. through time-series analysis of expression changes, improved accuracy of health status estimation, realization of individually optimized services, and automation and efficiency of analysis work. Specific application fields include health management apps, remote medical support, health monitoring in nursing care settings, stress check services, and rehabilitation support.

The provision unit can propose individually customized exercise programs based on the user's health status. For example, if the user is lacking exercise, the provision unit proposes light stretching or walking programs. If the user needs strength training, the provision unit can provide training menus for the gym. Furthermore, if the user requires rehabilitation, the provision unit can propose specialized rehabilitation programs. Thus, by providing exercise programs according to the user's health status, the provision unit can support health maintenance. Specifically, the provision unit receives as input the health status determination result from the determination unit (e.g., labels and probability values such as “exercise deficiency risk: high”, “muscle weakness risk: medium”, “rehabilitation necessity: high”) and collaborates with an exercise program database (e.g., structured data including program ID, exercise type, intensity, required time, recommended target, necessary equipment, rehabilitation applicability). The provision unit executes SQL queries or vector searches according to the determination result, and extracts program candidates under conditions such as “low-intensity stretching”, “aerobic exercise”, “strength training”, “joint range of motion training”. The provision unit ranks the extracted exercise program candidates by considering the user's past exercise history, health checkup data, medical history, rehabilitation history, age, and physical fitness level, using a recommendation engine (e.g., collaborative filtering, content-based recommendation, or Transformer-based exercise program recommendation model). Example inputs to the AI model include (1) health status determination labels and scores, (2) past exercise history vectors (e.g., walking three times a week, frequency of strength training), and (3) rehabilitation history and medical history information. The AI model generates outputs such as “recommended exercise: walking 30 minutes (5 times a week)”, “recommended strength training: lower body strengthening program”, “recommended rehabilitation: knee joint range of motion training”. The provision unit automatically generates and presents detailed information of the finally selected exercise program (e.g., exercise procedure, precautions, recommended frequency, required equipment, video guide, implementation record function) on the user interface, with images or videos. Furthermore, the provision unit can record the user's exercise implementation status in real time and collaborate with functions such as feedback, progress management, and reminder notifications. The technical effects are that the provision unit achieves improved accuracy of personalized exercise program recommendations, improved user experience, efficient formation of exercise habits, and optimization of rehabilitation support by automatic collaboration between health status determination results and the exercise program database. Unlike conventional uniform exercise proposals or user-dependent program selection, optimal health support for each user is enabled by AI-based real-time health status analysis and automatic exercise program optimization. Specific application fields include health management apps, fitness support services, remote rehabilitation support, exercise guidance in nursing care settings, and corporate health management support.

The determination unit can estimate the user's emotion and adjust the wording and tone when communicating health status determination results to the user based on the estimated emotion. For example, if the user is relaxed, the determination unit communicates the result with polite wording including detailed explanations. If the user is tense, the determination unit can communicate the result in a concise and reassuring tone. Furthermore, if the user is in a hurry, the determination unit can provide a quick explanation focusing on the key points. Thus, by communicating with consideration for the user's emotion, the determination unit can promote user understanding and acceptance. Specifically, the determination unit receives as input the facial image feature vector (e.g., complexion score, swelling score) from the analysis unit and the output of the emotion estimation AI model (e.g., relaxation level 0.72, tension level 0.18, hurry level 0.54). The emotion estimation AI model takes as input facial image tensors (e.g., 128×128×3 pixels), audio waveform data (e.g., 2 seconds of 16 kHz audio), and text input (e.g., chat history or spoken content), and uses convolutional neural networks or Transformer-based multimodal models to output emotion labels and scores such as “relaxed”, “tense”, or “in a hurry” from image, audio, and text. According to the estimated emotion scores, the determination unit sends signals to the determination result generation module: if relaxation level is high, a detailed explanation (e.g., polite sentences including determination rationale, comparison with past data, reliability indicators) is generated; if tension level is high, only the main determination results (e.g., “Health status: Good”, “Caution: Swelling tendency”) are notified as concise text and icons, with reassuring comments added; if hurry level is high, only the key points (e.g., “No abnormality”, “Caution required”) are immediately notified as pop-ups, and detailed explanations are omitted. The determination unit dynamically adjusts the detail level, tone, layout, and font size of the notification content, and automatically generates a communication interface optimized for the user's emotional state. Furthermore, the determination unit can learn from the user's past feedback and display history to evolve individually optimized explanation patterns. The technical effects are that the determination unit achieves improved user experience, information comprehension, stress reduction, and acceptance of determination results by optimizing explanation of determination results in conjunction with emotion estimation AI. Unlike conventional uniform explanations or user-dependent switching, optimal information delivery for each user is enabled by AI-based real-time emotion estimation and automatic explanation optimization. Specific application fields include health management apps, remote medical support, health monitoring in nursing care settings, and risk assessment for insurance companies.

The imaging unit can automatically detect the contour and feature points of the user's face when capturing a facial photograph and propose the optimal imaging angle. For example, if the user's face is not facing forward, the imaging unit adjusts the camera position to prompt imaging from the front. If the user's face appears dark, the imaging unit can adjust the lighting for brighter imaging. Furthermore, if part of the user's face is obscured, the imaging unit can adjust the position so that the entire face is visible. Thus, by detecting the contour and feature points of the face, the imaging unit provides optimal imaging conditions and enables accurate analysis. Specifically, the imaging unit uses real-time face detection algorithms (e.g., MTCNN, MediaPipe Face Mesh) to extract facial landmarks (e.g., 68-point feature coordinates) from camera images. The imaging unit estimates the orientation of the face (yaw, pitch, roll angles), and if the face is not facing forward, displays guidance such as “Please turn your face slightly to the right” on the screen. Based on the detection results of the facial contour and feature points, the imaging unit automatically proposes the optimal imaging angle (e.g., front, slightly upward, slightly downward), allowing the user to adjust the orientation of the face according to the guide. For lighting adjustment, the imaging unit automatically controls the camera's exposure value and white balance, analyzes the brightness histogram of the face region, and adjusts the LED light or screen brightness for optimal brightness. If part of the face is obscured, the imaging unit determines the detection score of the face region and the status of missing feature points, and issues instructions such as “Please adjust your position so that your entire face is visible”. These processes are executed in real time within tens of milliseconds, enabling acquisition of high-quality facial images while minimizing user burden. Example inputs to the AI model include (1) camera image frames, (2) facial feature point coordinate vectors, and (3) face region brightness histograms. The imaging unit executes imaging condition optimization algorithms (e.g., decision tree, reinforcement learning-based imaging condition optimization model) based on this information to automatically determine the optimal imaging angle, lighting, and face position. The technical effects are that the imaging unit achieves improved facial image quality, improved analysis accuracy, and reduced user operation burden through automatic detection of facial contour and feature points and optimization of imaging conditions. Unlike conventional static imaging settings or user-dependent imaging, analysis accuracy and user experience are greatly improved by AI-based automatic optimization of face orientation, lighting, and face position. Specific application fields include health management apps, remote medical facial image acquisition, identity verification systems, and skincare advice.

The analysis unit can analyze the texture of the skin and the condition of pores in detail when analyzing a user's facial photograph and provide skincare advice. For example, the analysis unit analyzes the dryness of the skin and proposes appropriate skincare products if moisturizing is needed. It can also analyze pore clogging and propose cleansing methods or products. Furthermore, the analysis unit analyzes skin elasticity and provides anti-aging care advice. Thus, by analyzing the skin condition in detail, the analysis unit can provide appropriate skincare advice to the user. Specifically, the analysis unit takes high-resolution facial image tensors (e.g., 512×512×3 pixels) as input, and performs preprocessing such as face region detection, skin region extraction (e.g., U-Net-based segmentation), and noise removal (e.g., bilateral filter). The analysis unit uses high-resolution image recognition models such as convolutional neural networks (CNN) or Vision Transformer to extract multidimensional vectors of skin texture features (e.g., GLCM, LBP, wavelet features), pore size, density, clogging score, estimated skin moisture content, and skin elasticity indicators (e.g., texture uniformity, fine wrinkle and sagging detection). Example inputs to the AI model include (1) high-resolution skin image tensor, (2) skin region texture feature vector, and (3) pore detection map. The AI model generates outputs such as “dryness: high (score 0.81)”, “pore clogging: medium (score 0.54)”, “elasticity: low (score 0.32)”, and transmits them to the skincare advice generation module. According to the output features and scores, the analysis unit automatically generates recommendations for moisturizing care, cleansing methods, and anti-aging care advice (e.g., collagen production promotion, massage procedures). Furthermore, the analysis unit can refer to the user's past skin condition data and skincare history to evolve individually optimized advice. The technical effects are that the analysis unit achieves improved accuracy of skin condition analysis, personalized skincare advice, and automation and efficiency of analysis work through high-resolution image analysis and multidimensional feature extraction. Unlike conventional visual observation or uniform advice provision, optimal skincare support for each user is enabled by AI-based automatic analysis and optimization of skin texture, pores, and elasticity. Specific application fields include health management apps, skincare advice, remote medical support, and dermatology diagnostic support.

The provision unit can estimate the user's emotion and adjust the timing of providing health-related information based on the estimated emotion of the user. For example, if the user is relaxed, the provision unit selects a timing to provide detailed information. If the user is busy, the provision unit can select a timing to provide concise information. Furthermore, if the user is feeling stressed, the provision unit can provide information at a timing when the user can relax. Thus, by providing information at a timing considerate of the user's emotion, the provision unit can improve the user's receptiveness. Specifically, the provision unit receives as input the health status determination result from the determination unit and the output of the emotion estimation AI model (e.g., relaxation level 0.68, tension level 0.21, stress level 0.54). The emotion estimation AI model uses facial image tensors, audio waveform data, and text input, and outputs emotion labels and scores using CNN or Transformer-based multimodal models. According to the estimated emotion scores, the provision unit sends signals to the information provision timing control module: if relaxation level is high, detailed health information and recommended actions are notified at times when the user is calm (e.g., nighttime, holidays); if busyness or stress level is high, concise information is immediately notified as a pop-up or notification is delayed until a relaxing timing. Information provision timing is dynamically adjusted by also considering the user's past information browsing history and notification response tendencies. Furthermore, the provision unit automatically selects the notification means (e.g., in-app notification, push notification, email) according to emotional state and usage trends. The technical effects are that the provision unit achieves improved user experience, information comprehension, stress reduction, and promotion of healthy behavior by optimizing information provision timing in conjunction with emotion estimation AI. Unlike conventional uniform notifications or user-dependent notification settings, optimal information delivery for each user is enabled by AI-based real-time emotion estimation and automatic timing optimization. Specific application fields include health management apps, remote medical support, health monitoring in nursing care settings, skincare advice, and identity verification systems.

The determination unit can collect the user's lifestyle data in real time when determining health status and reflect it in the analysis. For example, the determination unit collects meal content in real time and evaluates nutritional balance. It can also collect exercise amount in real time and determine exercise deficiency or excessive exercise. Furthermore, the determination unit collects sleep data in real time and evaluates sleep quality. Thus, by reflecting real-time lifestyle data in the analysis, the determination unit can determine health status more accurately. Specifically, the determination unit accumulates meal records (e.g., calorie intake, nutrient balance, meal frequency, meal timing), exercise records (e.g., step count, calories burned, exercise type, exercise frequency), and sleep records (e.g., bedtime and wake-up time, sleep duration, sleep efficiency, proportion of deep sleep) obtained from user terminals or wearable devices in a structured database in real time. The determination unit integrates facial image feature vectors and health checkup values with lifestyle data, and inputs them into machine learning models (e.g., multilayer perceptron, random forest, time series analysis models) to improve the accuracy of health status determination. Example inputs to the AI model include (1) the latest facial image feature vector, (2) time-series vectors of meal, exercise, and sleep data for the past 24 hours, and (3) past health checkup values. The AI model generates determination labels and scores such as “nutritional balance: good”, “exercise deficiency risk: high”, “sleep quality: low” from these inputs and generates health status determination results. The determination unit analyzes changes in real-time data over time to detect early signs of health deterioration or improvement. Furthermore, the determination unit can automatically detect abnormal values or sudden changes in lifestyle data and issue alert notifications to the user or medical professionals. The technical effects are that the determination unit achieves improved accuracy of health status determination, earlier anomaly detection, realization of personalized health management, and efficiency of data management and communication load by integrating real-time lifestyle data. Unlike conventional single-point data determination or uniform determinations, fine-grained health status determination is enabled by AI-based multidimensional and time-series data integration. Specific application fields include health management apps, remote medical support, risk assessment for insurance companies, health monitoring in nursing care settings, and corporate health management support.

The provision unit can propose individually customized meal plans based on the user's health status. For example, if the user wishes to diet, the provision unit proposes a low-calorie, nutritionally balanced meal plan. If the user wants to increase muscle strength, the provision unit can provide a high-protein meal plan. Furthermore, if the user has specific allergies, the provision unit can propose a meal plan that excludes allergens. Thus, by providing meal plans according to the user's health status, the provision unit can support health maintenance. Specifically, the provision unit receives as input the health status determination result from the determination unit (e.g., labels and probability values such as “diet wish”, “muscle strength increase wish”, “allergy: egg”) and collaborates with a meal plan database (e.g., structured data including plan ID, nutrient information, calories, protein amount, fat amount, allergen information, cooking time). The provision unit executes SQL queries or vector searches according to the determination result, and extracts meal plan candidates under conditions such as “low-calorie”, “high-protein”, “allergen exclusion”. The provision unit ranks the extracted meal plan candidates by considering the user's past meal history, preference data, and allergy information, using a recommendation engine (e.g., collaborative filtering, content-based recommendation, or Transformer-based meal plan recommendation model). Example inputs to the AI model include (1) health status determination labels and scores, (2) past meal history vectors, and (3) allergy information. The AI model generates outputs such as “recommended meal plan: low-calorie Japanese cuisine”, “recommended meal plan: high-protein Western cuisine”, “recommended meal plan: egg-free Chinese cuisine”. The provision unit automatically generates and presents detailed information of the finally selected meal plan (e.g., menu list, ingredients, quantities, cooking procedures, nutrition table, calorie calculation results) on the user interface, with images or videos. Furthermore, the provision unit can record the user's meal implementation status and collaborate with functions such as feedback, continuous support, and reminder notifications. The technical effects are that the provision unit achieves improved accuracy of personalized meal plan recommendations, improved user experience, and efficiency of meal management by automatic collaboration between health status determination results and the meal plan database. Unlike conventional uniform meal proposals or user-dependent plan selection, optimal health support for each user is enabled by AI-based real-time health status analysis and automatic meal plan optimization. Specific application fields include health management apps, remote nutrition guidance, meal management in nursing care settings, and corporate health management support.

The analysis unit can estimate the user's emotion and adjust the feedback method of the analysis result based on the estimated emotion of the user. For example, if the user is relaxed, detailed analysis results are provided. If the user is tense, concise feedback that provides reassurance can be given. Furthermore, if the user is in a hurry, rapid feedback focusing on key points can be provided. In this way, by providing feedback that takes the user's emotion into account, the analysis unit can promote the user's understanding and acceptance. Specifically, the analysis unit inputs facial image tensors, audio waveform data, text input, etc. into an emotion estimation AI model, and a multimodal model based on CNN or Transformer outputs emotion labels such as “relaxed,” “tense,” “in a hurry,” and scores (e.g., relaxation level 0.68, tension level 0.21, hurry level 0.54). According to the estimated emotion scores, the analysis unit sends signals to the feedback generation module: when the relaxation level is high, detailed analysis results (e.g., all feature quantities of complexion, swelling, puffiness, wrinkles, scars, time-series change graphs, reliability indicators, comparison with past data, etc.) are displayed; when the tension level is high, only major health indicators (e.g., complexion score, swelling score, overall health level) are presented as concise text and icons, with reassuring comments added; when the hurry level is high, only the key points (e.g., “Health status: Good,” “Notes: None,” etc.) are presented immediately. The level of detail, layout, color scheme, font size, etc. of the feedback content are dynamically adjusted, and an interface optimized for the user's emotional state is automatically generated. Furthermore, the analysis unit can learn from the user's past feedback history and response tendencies to evolve individually optimized feedback patterns. As a technical effect, the analysis unit achieves improved user experience, enhanced information comprehension, reduced stress, and increased acceptance of analysis results by optimizing analysis result feedback in conjunction with emotion estimation AI. Unlike conventional uniform feedback or user-dependent display switching, real-time emotion estimation and automatic feedback optimization by AI enable optimal information delivery for each user. Specific application fields include health management apps, remote medical support, health monitoring in nursing care settings, skincare advice, and identity verification systems.

The provision unit can propose individually customized relaxation plans based on the user's health condition. For example, if the user is feeling stressed, it proposes relaxation plans such as yoga or meditation. If the user is feeling fatigued, it can provide plans for refreshing massages or spa treatments. Furthermore, if the user is experiencing insomnia, it can propose relaxation music or aromatherapy plans to promote better sleep. In this way, by providing relaxation plans tailored to the user's health condition, the provision unit can support the user's physical and mental refreshment. Specifically, the provision unit receives health condition determination results (e.g., labels or probability values such as “stress level: high,” “fatigue level: medium,” “insomnia tendency: present”) from the determination unit as input and collaborates with a relaxation plan database (e.g., structured data including plan ID, type of relaxation, recommended target, required time, necessary equipment, details of music, aroma, massage, etc.). According to the determination result, the provision unit executes SQL queries or vector searches to extract plan candidates under conditions such as “yoga,” “meditation,” “massage,” “spa,” “relaxation music,” “aromatherapy,” etc. For the extracted plan candidates, the user's past relaxation history, preference data, and health condition are considered, and a recommendation engine (e.g., collaborative filtering, content-based recommendation, or a Transformer-based relaxation plan recommendation model) is used to rank the optimal plans. Examples of input to the AI model include (1) health condition determination labels/scores, (2) vectors of past relaxation history, and (3) indicators such as sleep, stress, and fatigue. The AI model generates outputs such as “Recommended plan: Night yoga 30 minutes,” “Recommended plan: Aroma massage,” “Recommended plan: Sleep music,” etc. The provision unit automatically generates and presents the details of the finally selected relaxation plan (e.g., implementation procedure, recommended time, required equipment, music/aroma playback function, video guide, etc.) on the user interface, including images and videos. Furthermore, the provision unit can record the user's relaxation implementation status and link functions such as feedback, continued support, and reminder notifications. As a technical effect, the provision unit achieves improved accuracy of personalized relaxation plan recommendations, enhanced user experience, and more efficient measures against stress, fatigue, and insomnia through automatic linkage between health condition determination results and the relaxation plan database. Unlike conventional uniform relaxation proposals or user-dependent plan selection, real-time health condition analysis and automatic relaxation plan optimization by AI enable optimal health support for each user. Specific application fields include health management apps, stress care support, sleep improvement services, relaxation support in nursing care settings, and corporate health management support.

Below, the processing flow of Example of the Embodiment is briefly described. Specifically, the present system automatically executes the entire process from acquiring the user's facial image to determining health condition and providing personalized information. First, the user captures a facial photograph using the camera function of a smartphone, tablet, or similar device. The imaging unit acquires the facial image as an RGB image tensor (e.g., 224×224×3 pixels) and performs preprocessing such as face region detection (e.g., MTCNN or YOLO-based), brightness and contrast correction, and noise removal. The analysis unit inputs the preprocessed facial image into an image recognition model such as a convolutional neural network (CNN) or Vision Transformer and extracts features such as complexion, swelling, puffiness, wrinkles, and scars as a multidimensional vector. The determination unit inputs the extracted feature vector into a machine learning model (e.g., random forest, SVM, deep learning model) trained with paired data of facial images and diagnostic results accumulated in medical settings (e.g., more than 100,000 cases) and infers the correlation between the state of the face and disease risk. The determination unit outputs probability distributions such as “anemia risk: high (probability 0.76),” “kidney disease risk: medium (probability 0.54),” and performs threshold determination or rule-based branching. The provision unit receives the determination result and, via a health recipe provision service API or electronic commerce site API, automatically generates and presents recipes or gym information according to the user's health condition. Furthermore, the user's emotion estimation AI, past behavioral history, location information, and lifestyle data are integrated to optimize the content, display method, priority, and timing of information in a personalized manner. All these processes are executed fully automatically and at high speed, achieving significant improvements in determination accuracy, processing speed, and user experience compared to conventional manual visual determination and manual search. As a technical effect, the present system achieves improved accuracy of health condition estimation through automatic extraction of high-dimensional image features and linkage with medical databases, immediate provision of individually optimized services to users, and efficient data management and communication load. Specific application fields include health management apps, remote medical support, corporate health management support, risk assessment for insurance companies, and health monitoring in nursing care settings.

Step 1: The imaging unit captures a facial photograph of the user. It is important to capture the user's facial photograph so that the entire face is clearly visible. For example, capturing under natural light makes it easier to accurately recognize the complexion and state of the face. Step 2: The analysis unit analyzes the facial photograph captured by the imaging unit. The analysis unit automatically recognizes features such as complexion, swelling, puffiness, wrinkles, and scars. For example, if the complexion is pale or there are dark circles under the eyes, information related to health condition is extracted. Step 3: The determination unit makes a determination using medical data based on the state of the face analyzed by the analysis unit. Medical data includes, for example, hospital medical records, health checkup results, and genetic information. The determination unit determines the correlation between the state of the face and diseases based on data accumulated in medical settings. For example, if the complexion is pale, it is determined that there is a possibility of anemia, and if there is swelling, it may suggest kidney problems. Step 4: The provision unit provides the determination result obtained by the determination unit to the user. For example, the provision unit provides a health recipe by utilizing a health recipe provision service based on the determination result. The provision unit can also inform the user of a nearby gym by utilizing an electronic commerce site based on the determination result. Specifically, in Step 1, the imaging unit acquires the facial image as an RGB image tensor (e.g., 224×224×3 pixels) and performs preprocessing such as face region detection (e.g., MTCNN or YOLO-based), brightness and contrast correction, and noise removal. In Step 2, the analysis unit inputs the preprocessed facial image into an image recognition model such as a convolutional neural network (CNN) or Vision Transformer and extracts features such as complexion, swelling, puffiness, wrinkles, and scars as a multidimensional vector. In Step 3, the determination unit inputs the extracted feature vector into a machine learning model (e.g., random forest, SVM, deep learning model) trained with paired data of facial images and diagnostic results accumulated in medical settings (e.g., more than 100,000 cases) and infers the correlation between the state of the face and disease risk. The determination unit outputs probability distributions such as “anemia risk: high (probability 0.76),” “kidney disease risk: medium (probability 0.54),” and performs threshold determination or rule-based branching. In Step 4, the provision unit receives the determination result and, via a health recipe provision service API or electronic commerce site API, automatically generates and presents recipes or gym information according to the user's health condition. Furthermore, the user's emotion estimation AI, past behavioral history, location information, and lifestyle data are integrated to optimize the content, display method, priority, and timing of information in a personalized manner. All these processes are executed fully automatically and at high speed, achieving significant improvements in determination accuracy, processing speed, and user experience compared to conventional manual visual determination and manual search. As a technical effect, the present system achieves improved accuracy of health condition estimation through automatic extraction of high-dimensional image features and linkage with medical databases, immediate provision of individually optimized services to users, and efficient data management and communication load. Specific application fields include health management apps, remote medical support, corporate health management support, risk assessment for insurance companies, and health monitoring in nursing care settings.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned imaging unit, analysis unit, determination unit, and provision unit is implemented, for example, by at least one of a smart device 14 and a data processing apparatus 12. For example, the imaging unit is implemented by a camera 42 of the smart device 14 and captures a facial photograph of a user. The analysis unit is implemented, for example, by a specific processing unit 290 of the data processing apparatus 12 and analyzes the captured facial photograph. The determination unit is implemented, for example, by the specific processing unit 290 of the data processing apparatus 12 and determines the correlation between the state of the face and diseases using medical data. The provision unit is implemented, for example, by a control unit 46A of the smart device 14 and provides the determination result to the user. The correspondence between each unit and the apparatus or control unit is not limited to the above examples and various modifications are possible.

### Second Embodiment

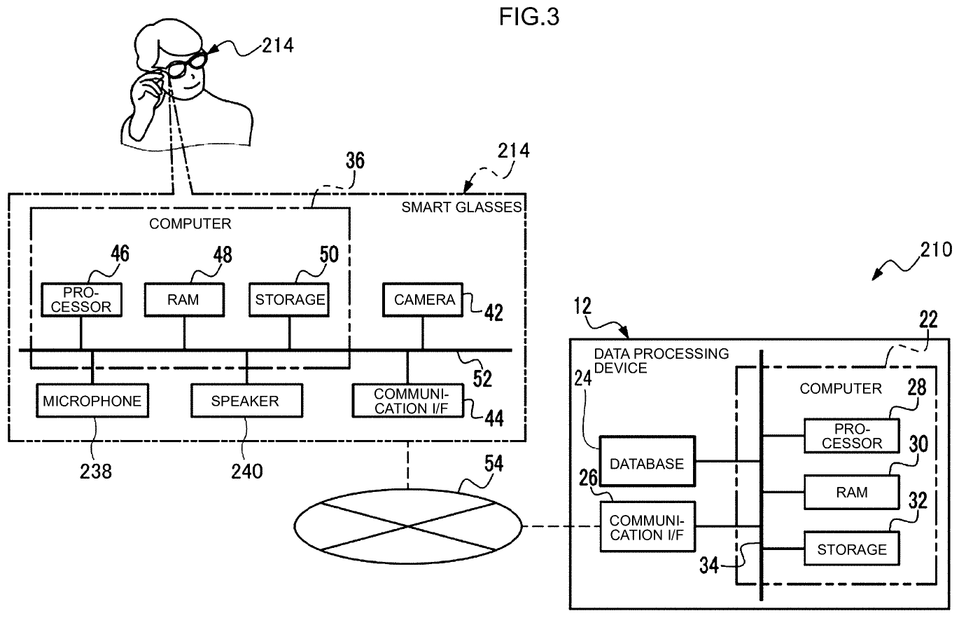

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

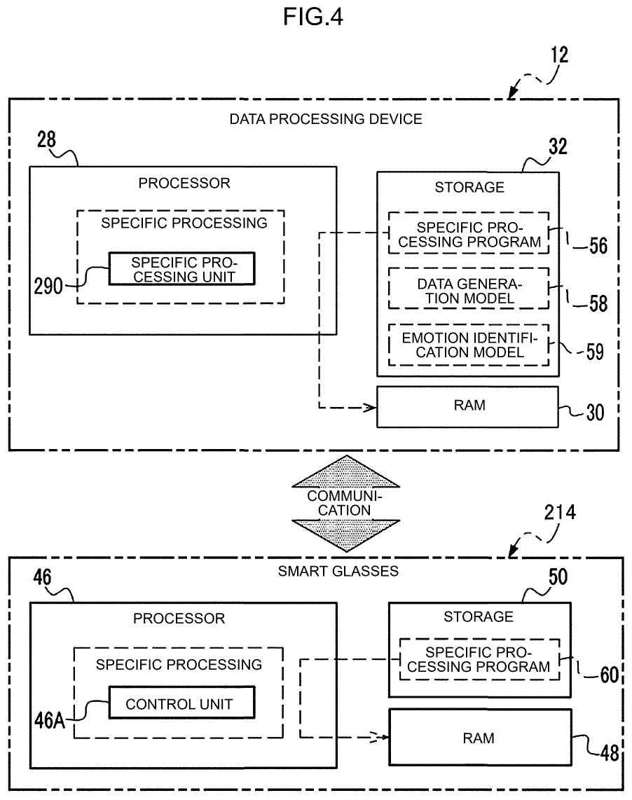

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned imaging unit, analysis unit, determination unit, and provision unit is implemented, for example, by at least one of smart glasses 214 and a data processing apparatus 12. For example, the imaging unit is implemented by a camera 42 of the smart glasses 214 and captures a facial photograph of a user. The analysis unit is implemented, for example, by a specific processing unit 290 of the data processing apparatus 12 and analyzes the captured facial photograph. The determination unit is implemented, for example, by the specific processing unit 290 of the data processing apparatus 12 and determines the correlation between the state of the face and diseases using medical data. The provision unit is implemented, for example, by a control unit 46A of the smart glasses 214 and provides the determination result to the user. The correspondence between each unit and the apparatus or control unit is not limited to the above examples and various modifications are possible.

### Third Embodiment

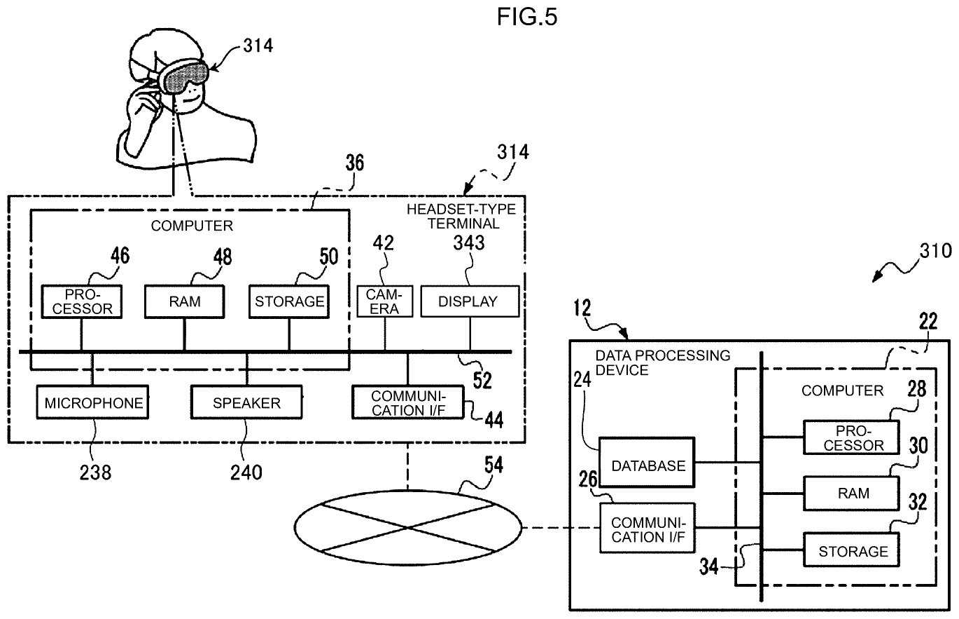

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

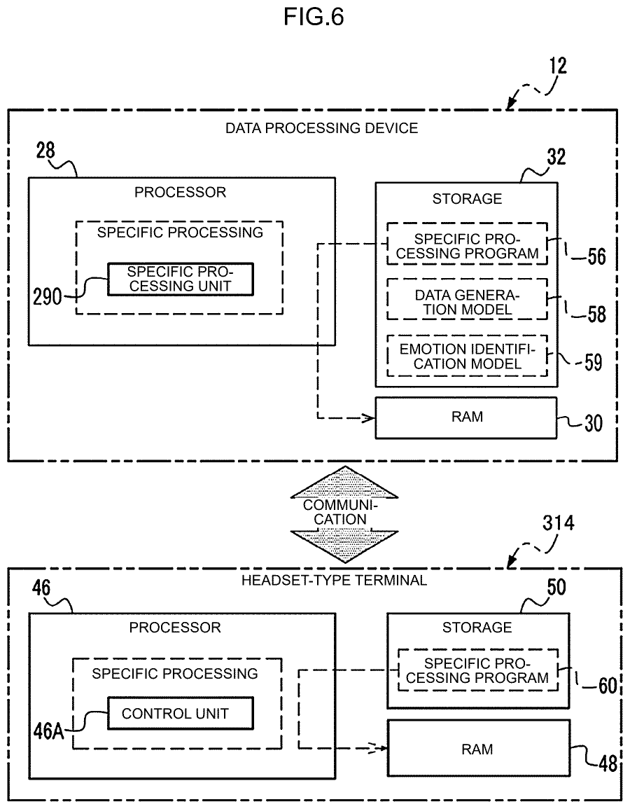

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned imaging unit, analysis unit, determination unit, and provision unit is implemented, for example, by at least one of a headset-type terminal 314 and a data processing apparatus 12. For example, the imaging unit is implemented by a camera 42 of the headset-type terminal 314 and captures a facial photograph of a user. The analysis unit is implemented, for example, by a specific processing unit 290 of the data processing apparatus 12 and analyzes the captured facial photograph. The determination unit is implemented, for example, by the specific processing unit 290 of the data processing apparatus 12 and determines the correlation between the state of the face and diseases using medical data. The provision unit is implemented, for example, by a control unit 46A of the headset-type terminal 314 and provides the determination result to the user. The correspondence between each unit and the apparatus or control unit is not limited to the above examples and various modifications are possible.

### Fourth Embodiment

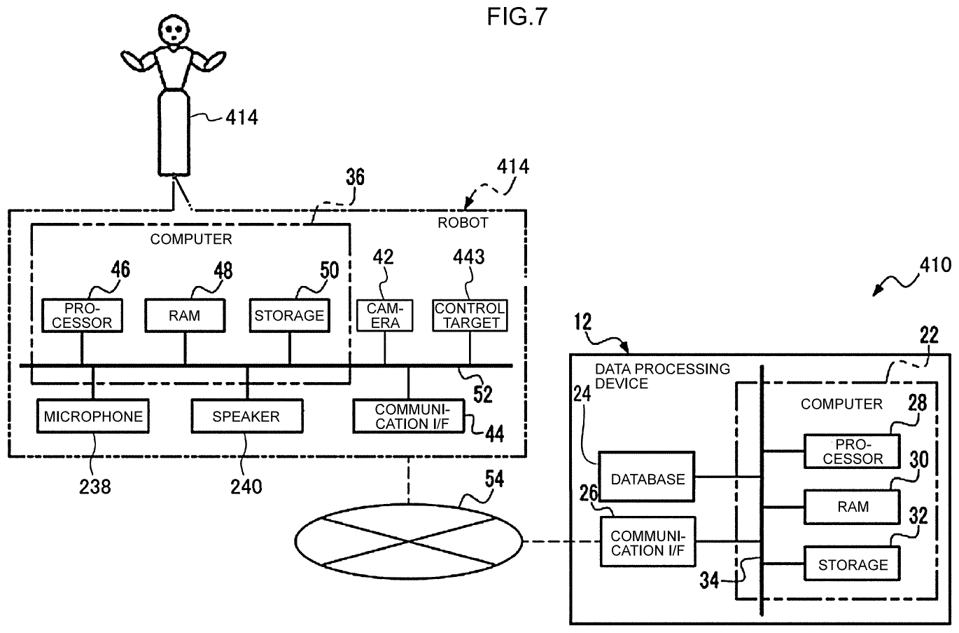

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

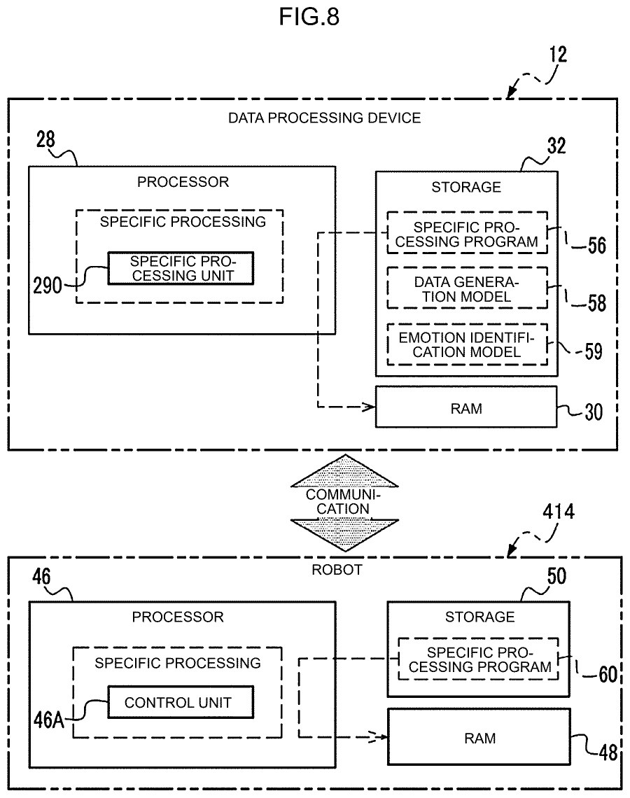

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned imaging unit, analysis unit, determination unit, and provision unit is implemented, for example, by at least one of a robot 414 and a data processing apparatus 12. For example, the imaging unit is implemented by a camera 42 of the robot 414 and captures a facial photograph of a user. The analysis unit is implemented, for example, by a specific processing unit 290 of the data processing apparatus 12 and analyzes the captured facial photograph. The determination unit is implemented, for example, by the specific processing unit 290 of the data processing apparatus 12 and determines the correlation between the state of the face and diseases using medical data. The provision unit is implemented, for example, by a control unit 46A of the robot 414 and provides the determination result to the user. The correspondence between each unit and the apparatus or control unit is not limited to the above examples and various modifications are possible.

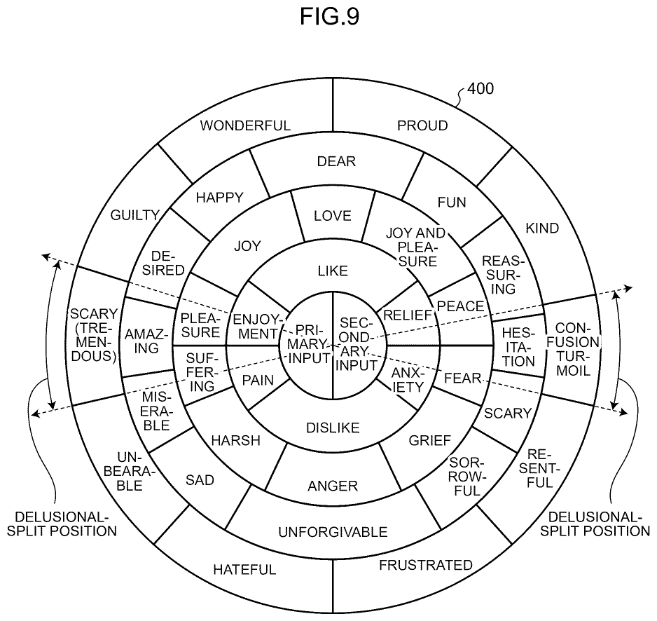

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

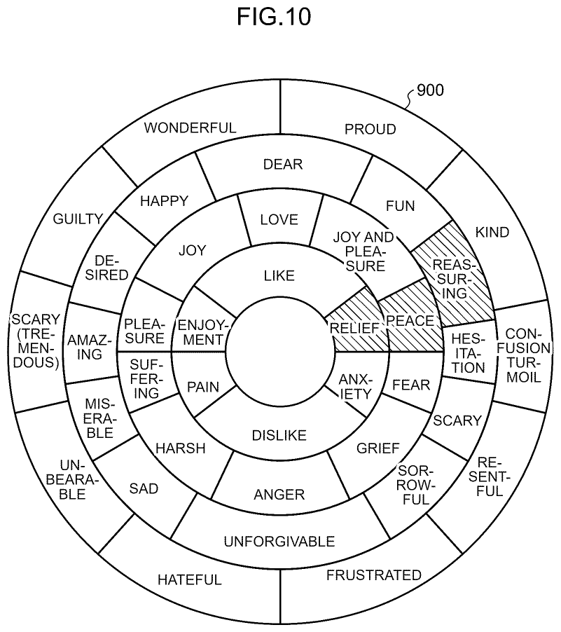

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: an imaging unit configured to capture a facial photograph of a user; an analysis unit configured to analyze the facial photograph captured by the imaging unit; a determination unit configured to make a determination using medical data based on the state of the face analyzed by the analysis unit; and a provision unit configured to provide the determination result obtained by the determination unit to the user.
(Supplementary Note 2) The system according to Supplementary Note 1, wherein the provision unit is configured to provide a health recipe by utilizing a health recipe provision service based on the determination result.
(Supplementary Note 3) The system according to Supplementary Note 1, wherein the provision unit is configured to inform the user of a nearby gym by utilizing an electronic commerce site based on the determination result.
(Supplementary Note 4) The system according to Supplementary Note 1, wherein the analysis unit is configured to automatically recognize features such as complexion, swelling, puffiness, wrinkles, and scars.
(Supplementary Note 5) The system according to Supplementary Note 1, wherein the determination unit is configured to determine the correlation between the state of the face and diseases based on data accumulated in medical settings.
(Supplementary Note 6) The system according to Supplementary Note 1, wherein the imaging unit is configured to estimate the user's emotion and adjust the imaging timing based on the estimated emotion of the user.
(Supplementary Note 7) The system according to Supplementary Note 1, wherein the imaging unit is provided with a function to automatically adjust the orientation and expression of the user's face during imaging.
(Supplementary Note 8) The system according to Supplementary Note 1, wherein the imaging unit uses light of different wavelengths during imaging to more accurately capture the condition of the user's skin.
(Supplementary Note 9) The system according to Supplementary Note 1, wherein the imaging unit is configured to estimate the user's emotion and adjust the imaging environment based on the estimated emotion of the user.
(Supplementary Note 10) The system according to Supplementary Note 1, wherein the imaging unit is configured to automatically select imaging settings by referring to the user's past imaging data during imaging.
(Supplementary Note 11) The system according to Supplementary Note 1, wherein the imaging unit is configured to propose an imaging location by considering the user's location information during imaging.
(Supplementary Note 12) The system according to Supplementary Note 1, wherein the analysis unit is configured to estimate the user's emotion and adjust the accuracy of analysis based on the estimated emotion of the user.
(Supplementary Note 13) The system according to Supplementary Note 1, wherein the analysis unit is configured to perform high-resolution image analysis to detect subtle changes in the face during analysis.
(Supplementary Note 14) The system according to Supplementary Note 1, wherein the analysis unit is configured to refer to the user's past health data during analysis to improve the accuracy of the analysis result.
(Supplementary Note 15) The system according to Supplementary Note 1, wherein the analysis unit is configured to estimate the user's emotion and adjust the display method of the analysis result based on the estimated emotion of the user.
(Supplementary Note 16) The system according to Supplementary Note 1, wherein the analysis unit is configured to refer to the user's lifestyle data during analysis to supplement the analysis result.
(Supplementary Note 17) The system according to Supplementary Note 1, wherein the analysis unit is configured to improve the accuracy of the analysis result based on the user's genetic information during analysis.
(Supplementary Note 18) The system according to Supplementary Note 1, wherein the determination unit is configured to estimate the user's emotion and adjust the reliability of the determination result based on the estimated emotion of the user.
(Supplementary Note 19) The system according to Supplementary Note 1, wherein the determination unit is configured to refer to multiple medical databases during determination to improve the accuracy of the determination result.
(Supplementary Note 20) The system according to Supplementary Note 1, wherein the determination unit is configured to refer to the user's past health checkup results during determination to supplement the determination result.
(Supplementary Note 21) The system according to Supplementary Note 1, wherein the determination unit is configured to estimate the user's emotion and adjust the notification method of the determination result based on the estimated emotion of the user.
(Supplementary Note 22) The system according to Supplementary Note 1, wherein the determination unit is configured to refer to the health data of the user's family during determination to supplement the determination result.
(Supplementary Note 23) The system according to Supplementary Note 1, wherein the determination unit is configured to improve the accuracy of the determination result based on the user's living environment data during determination.
(Supplementary Note 24) The system according to Supplementary Note 1, wherein the provision unit is configured to estimate the user's emotion and adjust the content of the information provided based on the estimated emotion of the user.
(Supplementary Note 25) The system according to Supplementary Note 1, wherein the provision unit is configured to refer to the user's past behavioral history during provision to provide optimal information.
(Supplementary Note 26) The system according to Supplementary Note 1, wherein the provision unit is configured to provide customized advice based on the user's current health condition during provision.
(Supplementary Note 27) The system according to Supplementary Note 1, wherein the provision unit is configured to estimate the user's emotion and determine the priority of the information provided based on the estimated emotion of the user.
(Supplementary Note 28) The system according to Supplementary Note 1, wherein the provision unit is configured to propose optimal services by considering the user's geographic location information during provision.
(Supplementary Note 29) The system according to Supplementary Note 1, wherein the provision unit is configured to analyze the user's social media activity during provision to provide relevant information.

What is claimed is:

1. A system comprising:
circuitry configured to:
receive, via a communication interface coupled to a packet-switched network, image data captured by a client terminal;
extract, by applying an image recognition model comprising a convolutional neural network to the image data, a multidimensional feature vector representing a plurality of attributes of the image data;
generate inference data by applying a machine learning model to the multidimensional feature vector, the machine learning model correlating the multidimensional feature vector with reference data stored in a database to produce a probability distribution associated with the plurality of attributes; and
transmit, via the communication interface, the inference data to the client terminal.

2. The system according to claim 1, wherein the inference data comprises a recommendation generated by querying a service database based on the probability distribution, and the circuitry is further configured to transmit the recommendation together with the inference data to the client terminal.

3. The system according to claim 1, wherein the inference data comprises location-based information generated by correlating the probability distribution with geographic coordinates received from the client terminal.

4. The system according to claim 1, wherein extracting the multidimensional feature vector comprises detecting a region of interest within the image data, normalizing the detected region, and applying the convolutional neural network to the normalized region to output a numerical vector representing at least one of hue, brightness, saturation, contour expansion, asymmetry, or texture depth.

5. The system according to claim 1, wherein the machine learning model comprises at least one of a multilayer perceptron, a random forest, a support vector machine, or a deep neural network trained on paired data of image feature vectors and labeled outcomes stored in the database.

6. The system according to claim 1, wherein the circuitry is further configured to estimate an emotion of a user based on biometric sensor data received from the client terminal, and adjust a capture timing parameter transmitted to the client terminal based on the estimated emotion, such that when the estimated emotion indicates relaxation, the circuitry transmits a capture command, and when the estimated emotion indicates tension, the circuitry transmits a delay instruction.

7. The system according to claim 1, wherein the circuitry is further configured to transmit orientation guidance data to the client terminal based on landmark coordinates extracted from the image data, the orientation guidance data comprising at least one of a yaw correction value, a pitch correction value, or a roll correction value.

8. The system according to claim 1, wherein the circuitry is further configured to receive the image data captured under a plurality of different wavelengths, and extract, for each wavelength, a wavelength-specific feature vector, and concatenate the wavelength-specific feature vectors into the multidimensional feature vector.

9. The system according to claim 1, wherein the circuitry is further configured to estimate an emotion of a user based on biometric sensor data received from the client terminal, and adjust an environmental parameter transmitted to the client terminal based on the estimated emotion, the environmental parameter comprising at least one of a brightness level or a color temperature setting.

10. The system according to claim 1, wherein the circuitry is further configured to retrieve past image data associated with the user from the database, and select a capture configuration based on the past image data, the capture configuration comprising at least one of a resolution setting, an exposure value, or a white balance parameter.

11. The system according to claim 1, wherein the circuitry is further configured to estimate an emotion of a user based on biometric sensor data received from the client terminal, and adjust a resolution of the image recognition model based on the estimated emotion, such that when the estimated emotion indicates relaxation, the circuitry applies a high-resolution analysis pipeline, and when the estimated emotion indicates urgency, the circuitry applies a reduced-resolution fast inference pipeline.

12. The system according to claim 1, wherein extracting the multidimensional feature vector comprises applying a high-resolution image analysis pipeline to detect sub-pixel level variations in the image data, the high-resolution image analysis pipeline comprising at least one of a multi-scale tensor analysis, a wavelet transform, or a region segmentation model.

13. The system according to claim 1, wherein the circuitry is further configured to retrieve historical feature vectors associated with the user from the database, and apply a time-series analysis model to the historical feature vectors and the multidimensional feature vector to calculate a reliability index for the inference data.

14. The system according to claim 1, wherein the circuitry is further configured to estimate an emotion of a user based on biometric sensor data received from the client terminal, and adjust a display format of the inference data based on the estimated emotion, such that when the estimated emotion indicates relaxation, the circuitry generates the inference data in a detailed format, and when the estimated emotion indicates stress, the circuitry generates the inference data in a simplified format.

15. The system according to claim 1, wherein the circuitry is further configured to integrate the multidimensional feature vector with supplementary structured data received from the client terminal, the supplementary structured data comprising at least one of activity log data or environmental sensor data, and generate the inference data based on the integrated data.

16. The system according to claim 1, wherein the circuitry is further configured to query a plurality of reference databases, each storing reference data from a different source, and generate the inference data by applying an ensemble learning model that weights outputs from each of the plurality of reference databases based on a recency metric and a reliability metric.

17. The system according to claim 1, wherein the circuitry is further configured to retrieve historical assessment records associated with the user from the database, and supplement the inference data based on the historical assessment records by adjusting a threshold of the probability distribution according to a trend detected in the historical assessment records.

18. A system comprising:
a communication interface configured to communicate with a client terminal via a packet-switched network;
a memory storing an image recognition model comprising a convolutional neural network and a machine learning model, each obtained by machine learning;
a database storing reference data comprising paired image feature vectors and labeled outcomes; and
circuitry comprising at least one of a CPU, a GPU, or a TPU, the circuitry configured to:
receive, from the client terminal via the communication interface, image data representing a region of interest captured by an image sensor of the client terminal;
detect the region of interest within the image data and normalize the detected region by applying at least one of brightness correction, contrast correction, or noise removal;
extract a multidimensional feature vector from the normalized region by inputting the normalized region into the image recognition model, the multidimensional feature vector representing a plurality of attributes comprising at least one of hue, brightness, saturation, contour expansion, asymmetry, or texture depth;
generate inference data by inputting the multidimensional feature vector into the machine learning model, the machine learning model comprising at least one of a multilayer perceptron, a random forest, or a support vector machine, and correlating the multidimensional feature vector with the reference data stored in the database to produce a probability distribution associated with the plurality of attributes; and
transmit the inference data to the client terminal via the communication interface and the packet-switched network, the inference data causing the client terminal to present the inference data to a user.

19. The system according to claim 18, wherein the memory further stores an emotion identification model, and wherein the circuitry is further configured to estimate an emotion of the user by inputting biometric sensor data received from the client terminal into the emotion identification model, and adjust at least one of: a resolution of the image recognition model, a threshold of the probability distribution, or a display format of the inference data, based on the estimated emotion.

20. A method performed by a system comprising a communication interface, a memory, a database, and circuitry, the method comprising:
receiving, from a client terminal via the communication interface and a packet-switched network, image data captured by an image sensor of the client terminal;
extracting, by applying an image recognition model stored in the memory and comprising a convolutional neural network, a multidimensional feature vector representing a plurality of attributes of the image data;
generating inference data by applying a machine learning model stored in the memory to the multidimensional feature vector, the machine learning model correlating the multidimensional feature vector with reference data stored in the database to produce a probability distribution associated with the plurality of attributes; and
transmitting the inference data to the client terminal via the communication interface and the packet-switched network.
